(19) United States
(12) Patent Application Publication
(10) Pub. No.: US 2015/0090258 A1
(43) Pub. Date: Apr. 2, 2015

(54) VENTILATOR-INITIATED PROMPT OR SETTING REGARDING DETECTION OF ASYNCHRONY DURING VENTILATION

(71) Applicant: Covidien LP, Boulder, CO (US)

(72) Inventors: Gary Milne, Louisville, CO (US); Peter R. Doyle, Vista, CA (US); Gardner Kimm, Carlsbad, CA (US)

(73) Assignee: Covidien LP, Boulder, CO (US)

(21) Appl. No.: 14/501,406

(22) Filed: Sep. 30, 2014

Related U.S. Application Data
(60) Provisional application No. 61/885,125, filed on Oct. 1, 2013.

(51) Int. Cl. A61M 16/00

(52) U.S. Cl. CPC A61M 16/0051; A61M 16/0069; A61M 16/0003; A61M 2230/42; A61M 2016/0027; A61M 2205/502; A61M 2230/06; A61M 2016/003; A61M 2205/15
USPC 128/202.22; 128/204.23

(57) ABSTRACT

Systems and methods are provided for monitoring and evaluating diverse ventilatory parameters to detect an asynchrony and may issue notifications and recommendations suitable for a patient to the clinician when asynchrony, such as an ineffective effort and an auto-trigger, is implicated. The suitable notifications and recommendations may further be provided in a hierarchical format such that the clinician may selectively access summarized and/or detailed information regarding the presence of asynchrony. In more automated systems, recommendations may be automatically implemented.

### INTRODUCTION

A ventilator is a device that mechanically helps patients breathe by replacing some or all of the muscular effort required to inflate and deflate the lungs. In recent years, there has been an accelerated trend towards an integrated clinical environment. That is, medical devices are becoming increasingly integrated with communication, computing, and control technologies. As a result, modern ventilatory equipment has become increasingly complex, providing for detection and evaluation of a myriad of ventilatory parameters. However, due to the sheer magnitude of available ventilatory data, many clinicians may not readily assess and evaluate the diverse ventilatory data to detect certain patient conditions and/or changes in patient conditions, such as ventilator asynchrony. Additionally, extended periods of asynchrony can increase the amount of time patient needs to be ventilated by the ventilator.

### Ventilator-Initiated Prompt or Setting Regarding Detection of Asynchrony During Ventilation of a Patient

This disclosure describes systems and methods for monitoring and evaluating ventilatory parameters, analyzing ventilatory data associated with those parameters, and providing useful notifications and/or recommendations to clinicians. Modern ventilators monitor, evaluate, and graphically represent a myriad of ventilatory parameters. However, many clinicians may not easily identify or recognize data patterns and correlations indicative of certain patient conditions, changes in patient condition, and/or effectiveness of ventilatory treatment. Further, clinicians may not readily determine appropriate ventilatory adjustments that may address certain patient conditions and/or the effectiveness of ventilatory treatment. Specifically, clinicians may not readily detect or recognize the presence of asynchrony.

According to embodiments, a ventilator may be configured to monitor and evaluate diverse ventilatory parameters to detect an asynchrony and may issue notifications and recommendations suitable for a patient to the clinician when asynchrony, such as an ineffective effort and an auto-trigger, is implicated. The suitable notifications and recommendations may further be provided in a hierarchical format such that the clinician may selectively access summarized and/or detailed information regarding the presence of asynchrony. In more automated systems, recommendations may be automatically implemented.

According to embodiments, ventilator-implemented methods for detecting asynchrony are provided. The methods include collecting data associated with ventilatory parameters and processing the collected ventilatory parameter data. The processing of the collected ventilatory parameter data comprises deriving ventilatory parameter data from the collected ventilatory parameter data. The processed ventilator parameter data includes at least one of a respiration rate and an expiratory time. The method also includes determining that an auto-trigger is implicated upon detecting that the processed ventilator parameter data breaches a received predetermined threshold. The method further includes issuing a smart prompt when the auto-trigger is implicated.

According to further embodiments, a ventilatory system for issuing a smart prompt when asynchrony is implicated during ventilation of a patient is provided. The system includes at least one processor and at least one memory. The at least one memory is communicatively coupled to the at least one processor and contains instructions that are executed by the at least one processor. The instructions include: detecting that an auto-trigger is implicated for the patient based on at least one of a respiration rate and an expiratory time; determining an appropriate notification message; determining an appropriate recommendation message; and issuing at least one of the appropriate notification message and the appropriate recommendation message.

According to embodiments, ventilator-implemented methods for detecting asynchrony are provided. The methods include collecting data associated with ventilatory parameters and processing the collected ventilatory parameter data. The processing of the collected ventilatory parameter determines at least one of a pressure integral during exhalation for a moving time window and a flow integral during exhalation for a moving time window. The method also includes determining that an ineffective effort is implicated upon detecting that the processed ventilator parameter data breaches a received predetermined threshold. The method further includes issuing a smart prompt when the ineffective effort is implicated.

In some embodiments, a ventilatory system for issuing a smart prompt when asynchrony is implicated during ventilation of a patient is disclosed. The ventilatory system includes: means for collecting data associated with ventilatory parameters; means for processing the collected ventilatory parameter data, wherein the step of processing the collected ventilatory parameter data comprises deriving ventilatory parameter data from the collected ventilatory parameter data, wherein the processed ventilator parameter data includes at least one of respiration rate and expiratory time; means for determining that an auto-trigger is implicated upon detecting that the processed ventilatory parameter data breaches a received predetermined threshold; and means for issuing a smart prompt when the ineffective effort is implicated.

In some embodiments, a ventilatory system for issuing a smart prompt when asynchrony is implicated during ventilation of a patient is disclosed. The ventilatory system includes: means for detecting an auto-trigger based on at least one of a monitored respiration rate and an expiratory time, means for identifying the current ventilator settings and secondary conditions, means for determining the appropriate notification message, means for determining the appropriate primary recommendation message and the appropriate secondary recommendation for the patient based on the identified ventilator settings and secondary conditions, and means for issuing the notification message, the primary recommendation message and the secondary recommendation message.

In some embodiments, a ventilatory system for issuing a smart prompt when asynchrony is implicated during ventilation of a patient is disclosed. The ventilatory system includes: means for collecting data associated with ventilatory parameters; means for processing the collected ventilatory parameter data, wherein the step of processing the collected ventilatory parameter data comprises deriving ventilatory parameter data from the collected ventilatory parameter data, wherein the processed ventilator parameter data includes at least one of a pressure integral during exhalation for a moving time window and a flow integral during exhalation for a moving time window; means for determining that an ineffective effort is implicated upon detecting that the processed ventilatory parameter data breaches a received predetermined threshold; and means for issuing a smart prompt when the ineffective effort is implicated.

In some embodiments, a ventilatory system for issuing a smart prompt when asynchrony is implicated during ventilation of a patient is disclosed. The ventilatory system includes: means for detecting an ineffective based on at least one of a pressure integral during exhalation for a moving time window and a flow integral during exhalation for a moving time window, means for identifying the current ventilator settings and secondary conditions, means for determining the appropriate notification message, means for determining the appropriate primary recommendation message and the appropriate secondary recommendation for the patient based on the identified ventilator settings and secondary conditions, and means for issuing the notification message, the primary recommendation message and the secondary recommendation message.

In some embodiments, a graphical user interface for displaying one or more prompts corresponding to a detected condition, a ventilator configured with a computer having a user interface including the graphical user interface for accepting commands and for displaying information is disclosed. The graphical user interface includes at least one window and one or more elements within the at least one window comprising at least one prompt element for communicating information regarding a detected auto-trigger based on a monitored respiration rate and/or expiratory time. The at least one prompt element further comprises at least one of a notification message and one or more recommendation messages. The notification message comprises one or more alerts associated with a detected auto-trigger. The one or more recommendation messages comprise one or more recommendations for mitigating the detected auto-trigger. The one or more recommendations comprise one or more of:
a recommendation to adjust trigger sensitivity to a less sensitive setting;
a recommendation to enable leak compensation;
a recommendation to change a trigger type;
a recommendation to check a patient circuit for condensate; and
a recommendation to check a seal of a patient interface.
   In some embodiments, the one or more recommendations are also based on an identified ventilator setting and/or a secondary condition.

In additional embodiments, a graphical user interface for displaying one or more prompts corresponding to a detected condition, a ventilator configured with a computer having a user interface including the graphical user interface for accepting commands and for displaying information is disclosed. The graphical user interface includes at least one window and one or more elements within the at least one window comprising at least one prompt element for communicating information regarding a detected ineffective effort based on a monitored a pressure integral during exhalation for a moving time window and/or a flow integral during exhalation for a moving time window. The at least one prompt element further comprises at least one of a notification message and one or more recommendation messages. The notification message comprises one or more alerts associated with a detected ineffective effort. The one or more recommendation messages comprise one or more recommendations for mitigating the detected ineffective effort. The one or more recommendations comprise one or more of:
a recommendation to shorten an inspiration time by increasing a peak flow setting or by changing a waveform shape to square;
a recommendation to lower a tidal volume setting;
a recommendation to check for causes of increased resistance;
a recommendation to check for a need to suction or consider a delivering a bronchodilator;
a recommendation to reduce an inspiratory sensitivity setting or to change to a more sensitive trigger type;
a recommendation to increase an expiratory sensitivity setting or to increase a rise time setting;
a recommendation to lower a pressure support setting;
a recommendation to correct a leak, to enable leak compensation, or to increase an expiratory sensitivity setting;
a recommendation to lower an inspiratory pressure setting; and
a recommendation to increase an expiratory sensitivity setting.
   In some embodiments, the one or more recommendations are also based on an identified ventilator setting and/or a secondary condition.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of described technology and are not meant to limit the scope of the claims in any manner, which scope shall be based on the claims appended hereto.

FIG. 1 is a diagram illustrating an embodiment of an exemplary ventilator connected to a human patient.

FIG. 2A is a block-diagram illustrating an embodiment of a ventilatory system for monitoring and evaluating ventilatory parameters associated with asynchrony.

FIG. 2B is a block-diagram illustrating an embodiment of the asynchrony detection module shown in FIG. 2A.

FIG. 3 is a flow chart illustrating an embodiment of a method for detecting an implication of asynchrony.

FIG. 4 is a flow chart illustrating an embodiment of a method for issuing a smart prompt upon detecting an implication of asynchrony.

FIG. 5 is an illustration of an embodiment of a graphical user interface displaying a smart prompt having a notification message.

FIG. 6 is an illustration of an embodiment of a graphical user interface displaying an expanded smart prompt having a notification message and one or more recommendation messages.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment for alerting and advising clinicians regarding detected patient conditions.

This disclosure describes systems and methods for monitoring and evaluating ventilatory parameters, analyzing ventilatory data associated with those parameters, and providing useful notifications and/or recommendations to clinicians. Modern ventilators monitor, evaluate, and graphically represent a myriad of ventilatory parameters. However, many clinicians may not easily identify or recognize data patterns and correlations indicative of certain patient conditions, changes in patient condition, and/or effectiveness of ventilatory treatment. Further, clinicians may not readily determine appropriate ventilatory adjustments that may address certain patient conditions and/or the effectiveness of ventilatory treatment. Specifically, clinicians may not readily detect or recognize the presence of asynchrony during ventilation of a patient.

According to embodiments, a ventilator may be configured to monitor and evaluate diverse ventilatory parameters to detect asynchrony and may issue suitable notifications and recommendations to the clinician when asynchrony is implicated. The suitable notifications and recommendations may further be provided in a hierarchical format such that the clinician may selectively access summarized and/or detailed information regarding the presence of asynchrony. In more automated systems, recommendations may be automatically implemented.

### Ventilator System

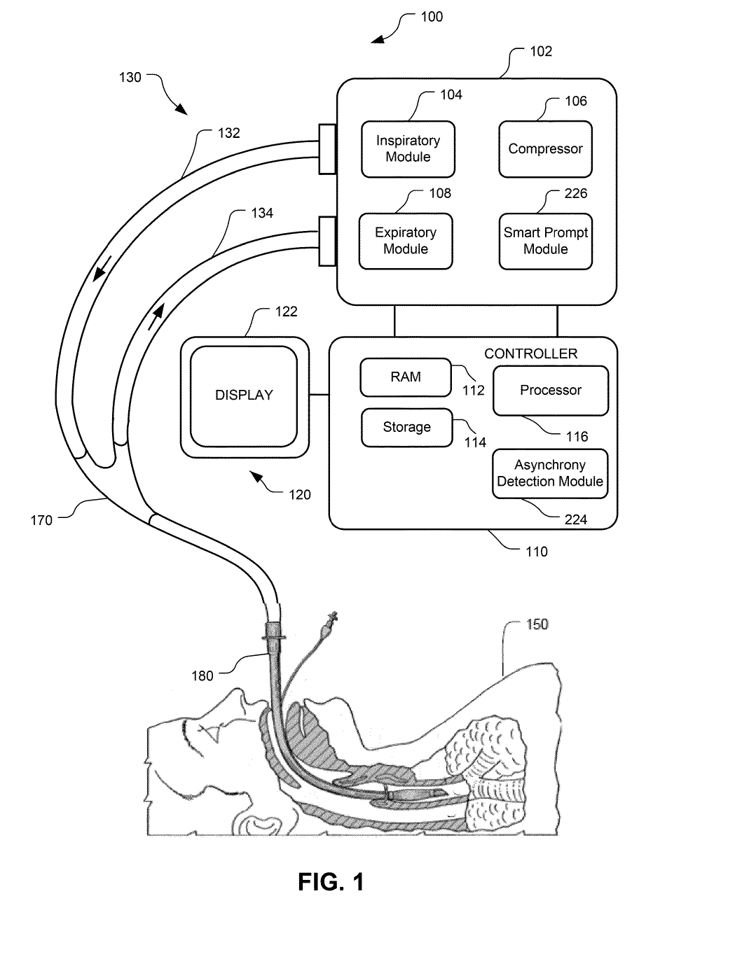

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

In some embodiments, the pneumatic system 102 includes a smart prompt module 226 as illustrated in FIG. 1. The smart prompt module 226 is described in further detail below. In other embodiments, the pneumatic system 102 includes an asynchrony module 224. The asynchrony module 224 is described in further detail below. In other embodiments, the pneumatic system 102 includes an asynchrony module 224. The pneumatic system 102 may include a variety of other components, including mixing modules, valves, sensors, tubing, accumulators, filters, etc.

Controller 110 is operatively coupled with pneumatic system 102, signal measurement and acquisition systems, and an operator interface 120 that may enable an operator to interact with the ventilator 100 (e.g., change ventilator settings, select operational modes, breath types, view monitored parameters, etc.). Controller 110 may include memory 112, one or more processors 116, storage 114, and/or other components of the type commonly found in command and control computing devices. In some embodiments, the controller 110 includes a smart prompt module 226 as illustrated in FIG. 1. In other embodiments, the controller 110 includes an asynchrony module 224. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilator 100 or between the ventilator 100 and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intranets or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

### Ventilator Components

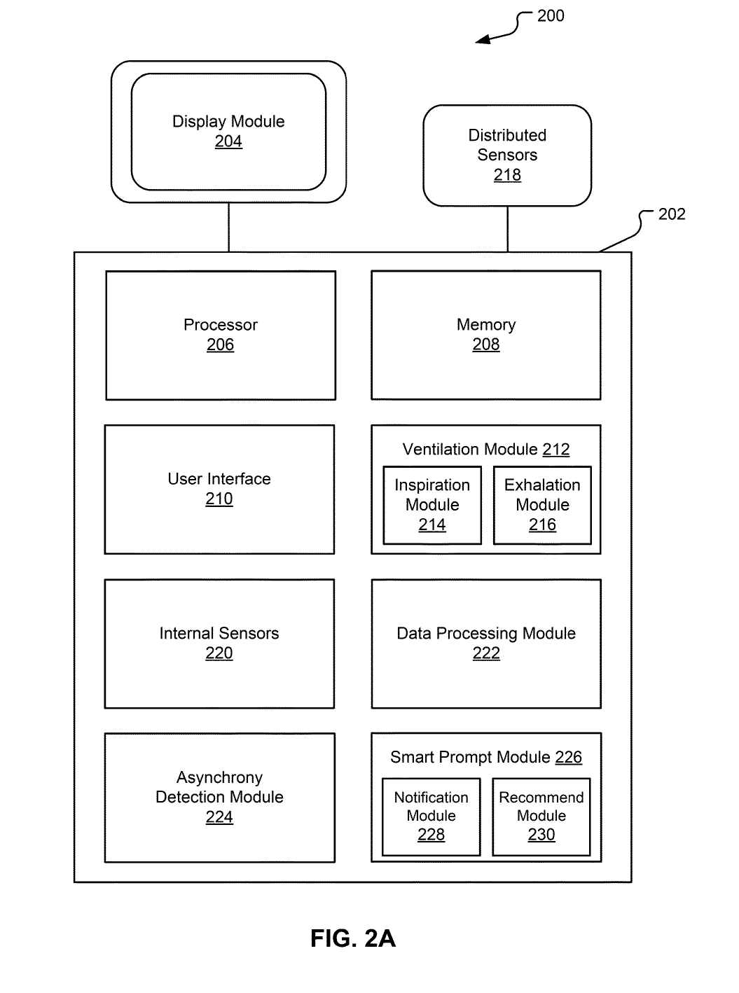

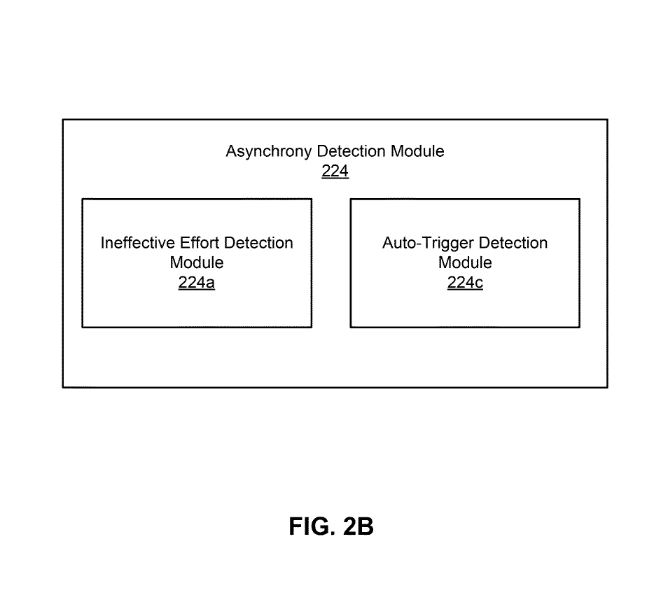

FIG. 2A is a block-diagram illustrating an embodiment of a ventilatory system 200 for monitoring and evaluating ventilatory parameters associated with asynchrony.

Ventilatory system 200 includes ventilator 202 with its various modules and components. That is, ventilator 202 may further include, inter alia, memory 208, one or more processors 206, user interface 210, and ventilation module 212 (which may further include an inspiration module 214 and an exhalation module 216). Memory 208 is defined as described above for memory 112. Similarly, the one or more processors 206 are defined as described above for one or more processors 116. Processors 206 may further be configured with a clock whereby elapsed time may be monitored by the ventilatory system 200.

The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., including ventilatory data, alerts, patient information, parameter settings, etc.). The elements may include controls, graphics, charts, tool bars, input fields, smart prompts, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician in the form of graphs, wave representations, pie graphs, or other suitable forms of graphic display. For example, one or more smart prompts may be displayed on the GUI and/or display module 204 upon detection of an implication of asynchrony by the ventilator. Additionally or alternatively, one or more smart prompts may be communicated to a remote monitoring system coupled via any suitable means to the ventilatory system 200.

### Equation of Motion

Ventilation module 212 may oversee ventilation of a patient according to prescribed ventilatory settings. By way of general overview, the basic elements impacting ventilation may be described by the following ventilatory equation (also known as the Equation of Motion):

Pₘ+Pᵥ=V_{T}/C+R*F

Here, Pₘ is a measure of muscular effort that is equivalent to the pressure generated by the muscles of a patient. If the patient's muscles are inactive, the Pₘ is equivalent to 0 cm H₂O. Pₘ is calculated using the following equation: Pₘ=elastance×volume+resistance×flow. During inspiration, Pᵥ represents the positive pressure delivered by a ventilator (generally in cm H₂O). V_{T} represents the tidal volume delivered, C refers to the respiratory compliance, R represents the respiratory resistance, and F represents the gas flow during inspiration (generally in liters per min (L/m)). Alternatively, during exhalation, the Equation of Motion may be represented as:

Pₐ+Pₜ=V_{TE}/C+R*F

Here, Pₐ represents the positive pressure existing in the lungs (generally in cm H₂O), Pₜ represents the transairway pressure, V_{TE} represents the tidal volume exhaled, C refers to the respiratory compliance, R represents the respiratory resistance, and F represents the gas flow during exhalation (generally in liters per min (L/m)).

### Pressure

For positive pressure ventilation, pressure at the upper airway opening (e.g., in the patient's mouth) is positive relative to the pressure at the body's surface (i.e., relative to the ambient atmospheric pressure to which the patient's body surface is exposed, about 0 cm H₂O). As such, when Pᵥ is zero, i.e., no ventilatory pressure is being delivered, the upper airway opening pressure will be equal to the ambient pressure (i.e., about 0 cm H₂O). However, when ventilatory pressure is applied, a pressure gradient is created that allows gases to flow into the airway and ultimately into the lungs of a patient during inspiration (or, inhalation).

According to embodiments, additional pressure measurements may be obtained and evaluated. For example, transairway pressure, Pₜ, which refers to the pressure differential or gradient between the upper airway opening and the alveoli, may also be determined Pₜ may be represented mathematically as:

Pₜ=P_{awo}−Pₐ

Where P_{awo} refers to the pressure in the upper airway opening, or mouth, and Pₐ refers to the pressure within the alveolar space, or the lungs (as described above). Pₜ may also be represented as follows:

Pₜ=F*R

Where F refers to flow and R refers to respiratory resistance, as described below.

Additionally, lung pressure or alveolar pressure, Pₐ, may be measured or derived. For example, Pₐ may be measured via a distal pressure transducer or other sensor near the lungs and/or the diaphragm. Alternatively, Pₐ may be estimated by measuring the plateau pressure, P_{Plat}, via a proximal pressure transducer or other sensor at or near the airway opening. Plateau pressure, P_{Plat}, refers to a slight plateau in pressure that is observed at the end of inspiration when inspiration is held for a period of time, sometimes referred to as an inspiratory hold or pause maneuver, or a breath-hold maneuver. That is, when inspiration is held, pressure inside the alveoli and mouth are equal (i.e., no gas flow). However, as a result of muscular relaxation and elastance of the lungs during the hold period, forces are exerted on the inflated lungs that create a positive pressure. This positive pressure is observed as a plateau in the pressure waveform that is slightly below the peak inspiratory pressure, P_{Peak}, prior to initiation of exhalation. As may be appreciated, for accurate measurement of P_{Plat}, the patient should be sedated or non-spontaneous (as muscular effort during the inspiratory pause may skew the pressure measurement). Upon determining P_{Plat} based on the pressure waveform or otherwise, P_{Plat} may be used as an estimate of Pₐ (alveolar pressure).

### Flow and Volume

Volume refers to the amount of gas delivered to a patient's lungs, usually in liters (L) or milliliters (ml). Flow refers to a rate of change in volume over time (F=ΔV/Δt). Flow is generally expressed in liters per minute (L/m or 1 μm) or milliliters per minute (mL/m) and, depending on whether gases are flowing into or out of the lungs, flow may be referred to as inspiratory flow or expiratory flow, respectively. According to embodiments, the ventilator may control the rate of delivery of gases to the patient, i.e., inspiratory flow, and may control the rate of release of gases from the patient, i.e., expiratory flow.

As may be appreciated, volume and flow are closely related. That is, where flow is known or regulated, volume may be derived based on elapsed time. Indeed, volume may be derived by integrating the flow waveform. According to embodiments, a tidal volume, V_{T}, may be delivered upon reaching a set inspiratory time (T_{I}) at set inspiratory flow. Alternatively, set V_{T} and set inspiratory flow may determine the amount of time required for inspiration, i.e., T_{I}.

### Respiratory Compliance

Additional ventilatory parameters that may be measured and/or derived may include respiratory compliance and respiratory resistance, which refer to the load against which the patient and/or the ventilator must work to deliver gases to the lungs. Respiratory compliance may be interchangeably referred to herein as compliance. Generally, compliance refers to a relative ease with which something distends and is the inverse of elastance, which refers to the tendency of something to return to its original form after being deformed. As related to ventilation, compliance refers to the lung volume achieved for a given amount of delivered pressure (C=ΔV/ΔP). Increased compliance may be detected when the ventilator measures an increased volume relative to the given amount of delivered pressure. Some lung diseases (e.g., acute respiratory distress syndrome (ARDS)) may decrease compliance and, thus, require increased pressure to inflate the lungs. Alternatively, other lung diseases may increase compliance, e.g., emphysema, and may require less pressure to inflate the lungs.

Additionally or alternatively, static compliance and dynamic compliance may be calculated. Static compliance, Cₛ, represents compliance impacted by elastic recoil at zero flow (e.g., of the chest wall, patient circuit, and alveoli). As elastic recoil of the chest wall and patient circuit may remain relatively constant, static compliance may generally represent compliance as affected by elastic recoil of the alveoli. As described above, P_{Plat} refers to a slight plateau in pressure that is observed after relaxation of pleural muscles and elastic recoil, i.e., representing pressure delivered to overcome elastic forces. As such, P_{Plat} provides a basis for estimating Cₛ as follows:

C_{S}=V_{T}/(P_{Plat}−EEP)

Where V_{T} refers to tidal volume, P_{Plat} refers to plateau pressure, and EEP refers to end-expiratory pressure, or baseline pressure (including PEEP and/or Auto-PEEP). Note that proper calculation of C_{S} depends on accurate measurement of V_{T} and P_{Plat}.

Dynamic compliance, C_{D}, is measured during airflow and, as such, is impacted by both elastic recoil and airway resistance. Peak inspiratory pressure, P_{Peak}, which represents the highest pressure measured during inspiration, i.e., pressure delivered to overcome both elastic and resistive forces to inflate the lungs, is used to calculate C_{D} as follows:

C_{D}=V_{T}/(P_{Peak}−EEP)

Where V_{T} refers to tidal volume, P_{Peak} refers to peak inspiratory pressure, and EEP refers to end-expiratory pressure. According to embodiments, ventilatory data may be more readily available for trending compliance of non-triggering patients than of triggering patients.

### Respiratory Resistance

Respiratory resistance refers to frictional forces that resist airflow, e.g., due to synthetic structures (e.g., endotracheal tube, expiratory valve, etc.), anatomical structures (e.g., bronchial tree, esophagus, etc.), or viscous tissues of the lungs and adjacent organs. Respiratory resistance may be interchangeably referred to herein as resistance. Resistance is highly dependent on the diameter of the airway. That is, a larger airway diameter entails less resistance and a higher concomitant flow. Alternatively, a smaller airway diameter entails higher resistance and a lower concomitant flow. In fact, decreasing the diameter of the airway results in an exponential increase in resistance (e.g., two-times reduction of diameter increases resistance by sixteen times). As may be appreciated, resistance may also increase due to a restriction of the airway that is the result of, inter alia, increased secretions, bronchial edema, mucous plugs, bronchospasm, and/or kinking of the patient interface (e.g., invasive endotracheal or tracheostomy tubes).

Airway resistance may further be represented mathematically as:

R=Pₜ/F

Where Pₜ refers to the transairway pressure and F refers to the flow. That is, Pₜ refers to the pressure necessary to overcome resistive forces of the airway. Resistance may be expressed in centimeters of water per liter per second (i.e., cm H₂O/L/s).

### Pulmonary Time Constant

As discussed above, compliance refers to the lung volume achieved for a given amount of delivered pressure (C=ΔV/ΔP). That is, stated differently, volume delivered is equivalent to the compliance multiplied by the delivered pressure (ΔV=C*ΔP). However, as the lungs are not perfectly elastic, a period of time is needed to deliver the volume ΔV at pressure ΔP. A pulmonary time constant, τ, may represent a time necessary to inflate or exhale a given percentage of the volume at delivered pressure ΔP. The pulmonary time constant, τ, may be calculated by multiplying the respiratory resistance by the respiratory compliance (τ=R*C) for a given patient and τ is generally represented in seconds, s. The pulmonary time constant associated with exhalation of the given percentage of volume may be termed an expiratory time constant and the pulmonary time constant associated with inhalation of the given percentage of volume may be termed an inspiratory time constant.

According to some embodiments, when expiratory resistance data is available, the pulmonary time constant may be calculated by multiplying expiratory resistance by compliance. According to alternative embodiments, the pulmonary time constant may be calculated based on inspiratory resistance and compliance. According to further embodiments, the expiratory time, T_{E}, should be equal to or greater than three (3) pulmonary time constants to ensure adequate exhalation. That is, for a triggering patient, expiratory time (T_{E}) (e.g., determined by trending T_{E} or otherwise) should be equal to or greater than 3 pulmonary time constants. For a non-triggering patient, set respiration rate (RR) should yield a T_{E} that is equal to or greater than 3 pulmonary time constants.

### Normal Resistance and Compliance

According to embodiments, normal respiratory resistance and compliance may be determined based on a patient's predicted body weight (PBW) (or ideal body weight (IBW)). That is, according to a standardized protocol or otherwise, patient data may be compiled such that normal respiratory resistance and compliance values and/or ranges of values may be determined and provided to the ventilatory system 200. That is, a manufacturer, clinical facility, clinician, or otherwise, may configure the ventilator with normal respiratory resistance and compliance values and/or ranges of values based on PBWs (or IBWs) of a patient population. Thereafter, during ventilation of a particular patient, respiratory resistance and compliance data may be trended for the patient and compared to normal values and/or ranges of values based on the particular patient's PBW (or IBW). According to embodiments, the ventilator may give an indication to the clinician regarding whether the trended respiratory resistance and compliance data of the particular patient falls into normal ranges. According to some embodiments, data may be more readily available for trending resistance and compliance for non-triggering patients than for triggering patients.

According to further embodiments, a predicted T_{E} may be determined based on a patient's PBW (or IBW). That is, according to a standardized protocol or otherwise, patient population data may be compiled such that predicted T_{E} values and/or ranges of values may be determined based on PBWs (or IBWs) of the patient population and provided to the ventilatory system 200. Actual (or trended) T_{E} for a particular patient may then be compared to the predicted T_{E}. As noted previously, increased resistance and/or compliance may result in an actual T_{E} that is longer than predicted T_{E}. However, when actual T_{E} is consistent with predicted T_{E}, this may indicate that resistance and compliance for the particular patient fall into normal ranges.

According to further embodiments, a normal pulmonary time constant, τ, may be determined based on a patient's PBW (or IBW). That is, according to a standardized protocol or otherwise, patient data may be compiled such that normal τ values and/or ranges of values may be determined based on PBWs (or IBWs) of a patient population and provided to the ventilatory system 200. A calculated ti may be determined for a particular patient by multiplying resistance by compliance (as described above, resistance and compliance data may be more readily available for a non-triggering patient). As the product of resistance and compliance results in τ, increased resistance and/or compliance may result in an elevated τ value. However, when the calculated ti value for the particular patient is consistent with the normal τ value, this may indicate that the resistance and compliance of the particular patient fall into normal ranges.

### Inspiration

Ventilation module 212 may further include an inspiration module 214 configured to deliver gases to the patient according to prescribed ventilatory settings. Specifically, inspiration module 214 may correspond to the inspiratory module 104 or may be otherwise coupled to source(s) of pressurized gases (e.g., air, oxygen, and/or helium), and may deliver gases to the patient. Inspiration module 214 may be configured to provide ventilation according to various ventilatory breath types, e.g., via volume-targeted, pressure-targeted, or via any other suitable breath types.

The various ventilator breath types operate in different modes such as mandatory, assist control (AC), mixed, and spontaneous modes. In some embodiments, the mode of operation is selected by the clinician. In other embodiments, the mode of operation is automatically determined by the ventilator.

During the spontaneous mode of operation, a breath type delivers inspiration and exhalation upon the detection of inspiratory and expiratory effort by the patient according to the parameters of the breath type. However, for safety measures, a breath type in a spontaneous mode may deliver inspiration and expiration after a predetermined amount of time passes to insure that the patient receives breathing gas in the event the patient stops making inspiratory and/or expiratory patient efforts. During the mandatory mode of operation, a breath type delivers inspiration and exhalation according to parameters of the breath type regardless of patient inspiratory and expiratory efforts.

During the assist control mode of operation, only mandatory breath types are delivered by the ventilator. During the assist control mode of operation, a breath is delivered or initiated based on the set respiration rate of the mandatory breath type unless a patient effort is detected. If a patient effort is detected during the assist control mode, the ventilator delivers or initiates the mandatory breath in response to the detected patient effort instead of the set respiration rate. However, the delivered mandatory breath still ends or terminates inspiration and begins exhalation based on the mandatory breath type settings. In other words, expiration begins based on the set mandatory breath type settings during the AC mode regardless of patient expiratory effort.

In the mixed mode, the ventilator delivers a mandatory breath type and a spontaneous breath type. In the mixed mode, a spontaneous breath type delivers inspiration and exhalation upon the detection of inspiratory and expiratory effort by the patient according to the parameters of the spontaneous breath type, or a mandatory breath type delivers inspiration and exhalation according to parameters of the mandatory breath type regardless of patient inspiratory and expiratory efforts. The determination as to whether a spontaneous or mandatory breath type is delivered is based upon the mandatory breath rate set by the clinician. Mandatory breath types will be delivered in order to achieve the mandatory breath rate setting. If the patient is breathing at a rate in excess of the mandatory breath rate setting, those excess breaths will be delivered based on the set spontaneous breath type. There are several different kinds of mixed modes. For example, a mixed mode may be a synchronized intermittent mandatory ventilation (SIMV) mixed mode. During SIMV, the delivery of mandatory breaths is synchronized with detected patient inspiratory efforts. Other types of mixed mode include BiLevel and intermittent mandatory ventilation.

Volume ventilation refers to various forms of volume-targeted ventilation that regulate volume delivery to the patient. Different types of volume ventilation are available depending on the specific implementation of volume regulation. For example, for volume-cycled ventilation, an end of inspiration is determined based on monitoring the volume delivered to the patient. Volume ventilation may include volume-control (VC), volume-targeted-pressure-control (VC+), or volume-support (VS) breath types. Volume ventilation may be accomplished by setting a target volume, or prescribed tidal volume, V_{T}, for delivery to the patient. According to embodiments, prescribed V_{T} and inspiratory time (T_{I}) may be set during ventilation start-up, based on the patient's PBW (or IBW). In this case, flow will be dependent on the prescribed V_{T} and set T_{I}. Alternatively, prescribed V_{T} and flow may be set and T_{I} may result. According to some embodiments, a predicted T_{E} may be determined based on normal respiratory and compliance values or value ranges based on the patient's PBW (or IBW). Additionally, a RR setting, generally in breaths/min, may be determined and configured. For a non-triggering patient, the set RR controls the timing for each inspiration. For a triggering patient, the RR setting applies if the patient stops triggering for some reason and/or the patient's triggered RR drops below a threshold level.

According to embodiments, during volume ventilation, as volume and flow are regulated by the ventilator, delivered V_{T}, flow waveforms (or flow traces), and volume waveforms may be constant and may not be affected by variations in lung or airway characteristics (e.g., respiratory compliance and/or respiratory resistance). Alternatively, pressure readings may fluctuate based on lung or airway characteristics. According to some embodiments, the ventilator may control the inspiratory flow and then derive volume based on the inspiratory flow and elapsed time. For volume-cycled ventilation, when the derived volume is equal to the prescribed V_{T}, the ventilator may initiate exhalation.

According to alternative embodiments, the inspiration module 214 may provide ventilation via a form of pressure ventilation. Pressure-targeted breath types may be provided by regulating the pressure delivered to the patient in various ways. For example, during pressure-cycled ventilation, an end of inspiration is determined based on monitoring the pressure delivered to the patient. Pressure ventilation may include a pressure-support (PS), a proportional assist (PA), tube compensation (TC), or a pressure-control (PC) breath type, for example. The proportional assist (PA) breath type provides pressure in proportion to the instantaneous patient effort during spontaneous ventilation and is based on the equation of motion. Pressure ventilation may also include various forms of bi-level (BL) pressure ventilation, i.e., pressure ventilation in which the inspiratory positive airway pressure (IPAP) is higher than the expiratory positive airway pressure (EPAP). Specifically, pressure ventilation may be accomplished by setting a target or prescribed pressure for delivery to the patient. During pressure ventilation, predicted T_{I} may be determined based on normal respiratory and compliance values and on the patient's PBW (or IBW). According to some embodiments, a predicted T_{E} may be determined based on normal respiratory and compliance values and based on the patient's PBW (or IBW). A respiratory rate (RR) setting may also be determined and configured. For a non-triggering patient, the set RR controls the timing for each inspiration. For a triggering patient, the RR setting applies if the patient stops triggering for some reason and/or patient triggering drops below a threshold RR level.

According to embodiments, during pressure ventilation, the ventilator may maintain the same pressure waveform at the mouth, P_{awo}, regardless of variations in lung or airway characteristics, e.g., respiratory compliance and/or respiratory resistance. However, the volume and flow waveforms may fluctuate based on lung and airway characteristics. As noted above, pressure delivered to the upper airway creates a pressure gradient that enables gases to flow into a patient's lungs. The pressure from which a ventilator initiates inspiration is termed the end-expiratory pressure (EEP) or “baseline” pressure. This pressure may be atmospheric pressure (about 0 cm H₂O), also referred to as zero end-expiratory pressure (ZEEP). However, commonly, the baseline pressure may be positive, termed positive end-expiratory pressure (PEEP). Among other things, PEEP may promote higher oxygenation saturation and/or may prevent alveolar collapse during exhalation. Under pressure-cycled ventilation, upon delivering the prescribed pressure the ventilator may initiate exhalation.

According to still other embodiments, a combination of volume and pressure ventilation may be delivered to a patient, e.g., volume-targeted-pressure-control (VC+) breath type. In particular, VC+ may provide benefits of setting a target V_{T}, while also allowing for monitoring variations in flow. In other embodiments, a positive feedback ventilation is delivered to the patient, e.g., a diaphragmatic electromyography adjusted (DEA) breath type, or an IE Sync™ breath type (owned by Covidien LP located at 6135 Gunbarrel Avenue in Boulder, Colo. 80301). As will be detailed further below, variations in flow may be indicative of various patient conditions. The use of an IE Synch or DEA breath type provides for more sensitive trigger detection for spontaneously breathing patients compared to other utilized breath types.

### Exhalation

Ventilation module 212 may further include an exhalation module 216 configured to release gases from the patient's lungs according to prescribed ventilatory settings. Specifically, exhalation module 216 may correspond to expiratory module 108 or may otherwise be associated with and/or controlling an expiratory valve for releasing gases from the patient. By way of general overview, a ventilator may initiate exhalation based on lapse of an inspiratory time setting (T_{I}) or other cycling criteria set by the clinician or derived from ventilator settings (e.g., detecting delivery of prescribed V_{T} or prescribed pressure based on a reference trajectory). Upon initiating the expiratory phase, exhalation module 216 may allow the patient to exhale by opening an expiratory valve. As such, exhalation is passive, and the direction of airflow, as described above, is governed by the pressure gradient between the patient's lungs (higher pressure) and the ambient surface pressure (lower pressure). Although expiratory flow is passive, it may be regulated by the ventilator based on the size of the expiratory valve opening. In some embodiments, exhalation is regulated based on a selected breath type.

Expiratory time (T_{E}) is the time from the end of inspiration until the patient triggers for a spontaneously breathing patient. The cycle detection for exhalation may be based on a selected trigger type. For a non-triggering patient, the T_{E} is the time from the end of inspiration until the next inspiration based on the set RR. In some cases, however, the time required to return to the functional residual capacity (FRC) or resting capacity of the lungs is longer than provided by T_{E} (e.g., because the patient triggers prior to fully exhaling or the set RR is too high for a non-triggering patient). According to embodiments, various ventilatory settings may be adjusted to better match the time to reach FRC with the time available to reach FRC. For example, increasing flow will shorten T_{E} thereby increasing the amount of time available to reach FRC. Alternatively, V_{T} may be decreased, resulting in less time required to reach FRC.

As may be further appreciated, at the point of transition between inspiration and exhalation, the direction of airflow may abruptly change from flowing into the lungs to flowing out of the lungs or vice versa depending on the transition. Stated another way, inspiratory flow may be measurable in the ventilatory circuit until P_{Peak} is reached, at which point flow is zero. Thereafter, upon initiation of exhalation, expiratory flow is measurable in the ventilatory circuit until the pressure gradient between the lungs and the body's surface reaches zero (again, resulting in zero flow). However, in some cases, as will be described further herein, expiratory flow may still be positive, i.e., measurable, at the end of exhalation (termed positive end-expiratory flow or positive EEF). In this case, positive EEF is an indication that the pressure gradient has not reached zero or, similarly, that the patient has not completely exhaled. Although a single occurrence of premature inspiration may not warrant concern, repeated detection of positive EEF may be indicative of Auto-PEEP.

### Ventilator Synchrony and Patient Triggering

According to some embodiments, the inspiration module 214 and/or the exhalation module 216 may be configured to synchronize ventilation with a spontaneously-breathing, or triggering, patient. That is, the ventilator may be configured to detect patient effort and may initiate a transition from exhalation to inspiration (or from inspiration to exhalation) in response. Triggering refers to the transition from exhalation to inspiration in order to distinguish it from the transition from inspiration to exhalation (referred to as cycling). Ventilation systems, depending on their breath type, may trigger and/or cycle automatically, or in response to a detection of patient effort, or both.

In the medical device field, “patient effort” is a term that can be used to describe many different patient parameters. To be clear, for the purposes of this document, the term “patient effort” shall be used herein to mean a patient's spontaneous attempt to initiate an inspiration or an exhalation as determined by an analysis of pressure, flow, volume, etc. measured by the ventilator. For example, a drop in pressure of greater than a threshold amount may be detected and identified as a single effort of the patient to initiate an inspiration. At times, the phrase “patient inspiratory effort” or “patient expiratory effort” will be used instead of patient effort to remind the reader that what is meant is an attempt by the patient to change the phase of respiratory cycle.

There are several different trigger types or systems and/methods utilized by the ventilator for detecting patient triggers and/or cycles. Once a breath type is selected, the trigger type utilized by the breath type for detecting patient effort may be selected. In some embodiments, the trigger type utilized by the breath type is automatically selected by the ventilator. In other embodiments, the trigger type utilized by the breath type is selected by the operator.

Any suitable type of triggering detection for determining a patient trigger may be utilized by the ventilator, such as nasal detection, diaphragm detection, and/or brain signal detection. Further, the ventilator may detect patient triggering via a pressure-monitoring method, a flow-monitoring method, direct or indirect measurement of neuromuscular signals, or any other suitable method. Internal sensors 220 and/or distributed sensors 218 suitable for this detection may include any suitable sensing device as known by a person of skill in the art for a ventilator. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight drop in circuit pressure. The slight drop in circuit pressure may indicate that the patient's respiratory muscles, Pₘ, are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, while gases are generally flowing out of the patient's lungs during exhalation, a drop in flow may occur as some gas is redirected and flows into the lungs in response to the slightly negative pressure gradient between the patient's lungs and the body's surface. Thus, when the ventilator detects a slight drop in flow below the bias flow by a predetermined threshold amount (e.g., 2 L/min below bias flow), it may interpret the drop as a patient trigger and may consequently initiate inspiration by delivering respiratory gases.

Further, in some embodiments, the trigger type may be an “active trigger type” or a “background trigger type”. The active trigger type determines when to deliver inspiration and/or expiration to the patient during ventilation by the ventilator and the ventilator actively delivers inspiration and/or expiration based on this determination. A background trigger type determines when to deliver inspiration and/or expiration to the patient during ventilation by the ventilator but the ventilator does not actively deliver inspiration and/or expiration based on this determination and is, therefore, merely running in the background. Any trigger type described herein may be an active trigger type or a background trigger type.

### Volume-Control Breath Type

In some embodiments, ventilation module 212 may further include an inspiration module 214 configured to deliver gases to the patient according to volume-control (VC). The VC breath type allows a clinician to set a respiratory rate and to select a volume to be administered to a patient during a mandatory breath. When using VC, a clinician sets a desired tidal volume, flow wave form shape, and an inspiratory flow rate or inspiratory time. These variables determine how much volume of gas is delivered to the patient and the duration of inspiration during each mandatory breath inspiratory phase. The mandatory breaths are administered according to the set respiratory rate.

For VC, when the delivered volume is equal to the prescribed tidal volume, the ventilator may initiate exhalation. Exhalation lasts from the time at which prescribed volume is reached until the start of the next ventilator mandated inspiration. This exhalation time is determined by the respiratory rate set by the clinician and any participation above the set rate by the patient. Upon the end of exhalation, another VC mandatory breath is given to the patient.

During VC, delivered volume and flow waveforms may remain constant and may not be affected by variations in lung or airway characteristics. Alternatively, pressure readings may fluctuate based on lung or airway characteristics. According to some embodiments, the ventilator may control the inspiratory flow and then derive volume based on the inspiratory flow and elapsed time.

In some embodiments, VC may also be delivered to a triggering patient. When VC is delivered to a triggering patient, the breath period (i.e. time between breaths) is a function of the frequency at which the patient is triggering breaths. That is, the ventilator will trigger the inhalation based upon the respiratory rate setting or the patient effort. If no patient effort is detected, the ventilator will deliver another mandatory breath at the predetermined respiratory rate. A patient-initiated mandatory (PIM) breath is a control breath that is triggered by the patient during a control mode such as VC or PC.

### Volume-Targeted-Pressure-Control Breath Type

In further embodiments, ventilation module 212 may further include an inspiration module 214 configured to deliver gases to the patient using a volume-targeted-pressure-control (VC+) breath type. The VC+ breath type is a combination of volume and pressure control breath types that may be delivered to a patient as a mandatory breath. In particular, VC+ may provide the benefits associated with setting a target tidal volume, while also allowing for variable flow. Variable flow may be helpful in meeting inspiratory flow demands for actively breathing patients.

As may be appreciated, when resistance increases it becomes more difficult to pass gases into and out of the lungs, decreasing flow. For example, when a patient is intubated, i.e., having either an endotracheal or a tracheostomy tube in place, resistance may be increased as a result of the smaller diameter of the tube over a patient's natural airway. In addition, increased resistance may be observed in patients with obstructive disorders, such as COPD, asthma, etc. Higher resistance may necessitate, inter alia, a higher inspiratory time setting for delivering a prescribed pressure or volume of gases, a lower respiratory rate resulting in a higher expiratory time for complete exhalation of gases.

Unlike VC, when the set inspiratory time is reached, the ventilator may initiate exhalation. Exhalation lasts from the end of inspiration until the beginning of the next inspiration. For a non-triggering patient, the expiratory time (T_{E}) is based on the respiratory rate set by the clinician. Upon the end of exhalation, another VC+ mandatory breath is given to the patient.

By controlling target tidal volume and allowing for variable flow, VC+ allows a clinician to maintain the volume while allowing the flow and pressure targets to fluctuate.

### Volume-Support Breath Type

In some embodiments, ventilation module 212 may further include an inspiration module 214 configured to deliver gases to the patient according to volume-support (VS) breath type. The VS breath type is utilized in the present disclosure as a spontaneous breath. VS is generally used with a triggering (spontaneously breathing) patient when the patient is ready to be weaned from a ventilator or when the patient cannot do all of the work of breathing on his or her own. When the ventilator senses patient inspiratory effort, the ventilator delivers a set tidal volume during inspiration. The tidal volume may be set and adjusted by the clinician. The patient controls the rate, inspiratory flow, and has some control over the inspiratory time. The ventilator then adjusts the pressure over several breaths to achieve the set tidal volume. When the machine senses a decrease in flow, or inspiration time reaches a predetermined limit, the ventilator determines that inspiration is ending. When delivered as a spontaneous breath, exhalation in VS lasts from a determination that inspiration is ending until the ventilator senses a next patient effort to breath.

### Pressure-Control Breath Type

In additional embodiments, ventilation module 212 may further include an inspiration module 214 configured to deliver gases to the patient according to the pressure-control (PC) breath type. PC allows a clinician to select a pressure to be administered to a patient during a mandatory breath. When using the PC breath type, a clinician sets a desired pressure, inspiratory time, and respiratory rate for a patient. These variables determine the pressure of the gas delivered to the patient during each mandatory breath inspiration. The mandatory breaths are administered according to the set respiratory rate.

For the PC breath type, when the inspiratory time is equal to the prescribed inspiratory time, the ventilator may initiate exhalation. Exhalation lasts from the end of inspiration until the next inspiration. Upon the end of exhalation, another PC mandatory breath is given to the patient.

During PC breaths, the ventilator may maintain the same pressure waveform at the mouth, regardless of variations in lung or airway characteristics, e.g., respiratory compliance and/or respiratory resistance. However, the volume and flow waveforms may fluctuate based on lung and airway characteristics.

In some embodiments, PC may also be delivered for triggering patients. When PC is delivered with triggering, the breath period (i.e. time between breaths) is a function of the respiratory rate of the patient. The ventilator will trigger the inhalation based upon the respiratory rate setting or the patient's trigger effort, but cycling to exhalation will be based upon elapsed inspiratory time. The inspiratory time is set by the clinician. The inspiratory flow is delivered based upon the pressure setting and patient physiology. Should the patient create an expiratory effort in the middle of the mandatory inspiratory phase, the ventilator will respond by reducing flow. If no patient effort is detected, the ventilator will deliver another mandatory breath at the predetermined respiratory rate.

PC with triggering overcomes some of the problems encountered by other mandatory breath types that use artificially set inspiratory flow rates. For example, if the inspiratory flow is artificially set lower than a patient's demand, the patient will feel starved for flow. This can lead to undesirable effects, including increased work of breathing. In addition, should the patient begin to exhale when using one of the traditional mandatory breath types, the patient's expiratory effort is ignored since the inspiratory flow is mandated by the ventilator settings.

### Pressure-Support Breath Type

In further embodiments, ventilation module 212 may further include an inspiration module 214 configured to deliver gases to the patient according to a pressure-support (PS) breath type. PS is a form of assisted ventilation and is utilized in the present disclosure during a spontaneous breath. PS is a patient triggered breath and is typically used when a patient is ready to be weaned from a ventilator or for when patients are breathing spontaneously but cannot do all the work of breathing on their own. When the ventilator senses patient inspiratory effort, the ventilator provides a constant pressure during inspiration. The pressure may be set and adjusted by the clinician. The patient controls the rate, inspiratory flow, and to an extent, the inspiratory time. The ventilator delivers the set pressure and allows the flow to vary. When the machine senses a decrease in flow, or determines that inspiratory time has reached a predetermined limit, the ventilator determines that inspiration is ending. When delivered as a spontaneous breath, exhalation in PS lasts from a determination that inspiration is ending until the ventilator senses a patient effort to breath.

### Proportional Assist Breath Type

In mechanical ventilation, a proportional assist (PA) breath type refers to a type of ventilation in which the ventilator acts as an inspiratory amplifier that provides pressure support based on the patient's work of breathing (WOB). The degree of amplification (the “support setting”) is set by an operator, for example, as a percentage based on the patient's WOB. In one implementation of a PA breath type, the ventilator may continuously monitor the patient's instantaneous inspiratory flow and instantaneous net lung volume, which are indicators of the patient's inspiratory WOB. These signals, together with ongoing estimates of the patient's lung compliance and lung resistance, allow the ventilator to compute a patient WOB and derive therefrom a target pressure to provide the support that assists the patient's inspiratory muscles to the degree selected by the operator as the support setting.

Various methods are known for calculation of patient WOB and any suitable method may be used. For example, methods exist that calculate patient WOB from sensors attached to the body to detect neural or muscular activity as well as methods that determine a patient WOB based on respiratory flow, respiratory pressure or a combination of both flow and pressure.

In a PA breath type, the patient's work of breathing, the elastic work of breathing component, and/or the resistive WOB component may be estimated by inputting measurements from various internal sensors 220 and/or distributed sensors 218 into the breathing algorithms. Typically, none of the instantaneous inspiratory pressure, the instantaneous flow, or the resulting volume are set by the caregiver. Because the PA breath type harmoniously links the ventilator to the patient, the patient effectively “drives” the ventilator. By appropriately setting the value of the proportionality (% support or support setting) control, the caregiver may effectively partition the total work of breathing between the patient and the ventilator.

### Tube Compensation Breath Type

A Tube Compensation (TC) breath type is similar to the PA breath type. The TC breath type delivers breathing gases to a spontaneously-breathing patient with the objective of reducing the patient's work of breathing imposed by an artificial airway. During a TC breath type, the ventilator compensates for the load associated with breathing through an endotracheal or tracheostomy tube. The TC breath type calculates a tube resistance based on the tube type (endotracheal or tracheostomy) and the tube's internal diameter (tube_{I.D.}), which are settings input by the clinician. A tube compensation pressure is then calculated by the ventilator during the TC breath type as a function of the patient's monitored flow, the calculated tube resistance, and a percent support setting (also known as support setting) input by the clinician. During inhalation, the ventilator during the TC breath type delivers the tube compensation pressure plus a set PEEP to the patient airway. Upon reaching an expiration sensitivity (E_{SENS}) setting (or other cycling criteria), the ventilator during the TC breath type initiates exhalation. As with other pressure-based breath types, the ventilator during the TC breath type does not target a set V_{T} or flow pattern.

### Expiratory Sensitivity

As discussed above, ventilation module 212 may oversee ventilation of a patient according to prescribed ventilatory settings. In one embodiment, the expiratory sensitivity (E_{SENS}) is set by a clinician or operator. According to embodiments, E_{SENS} sets the percentage of delivered peak inspiratory flow necessary to terminate inspiration and initiate exhalation. In some embodiments, the clinician or operator determines the E_{SENS} setting, which is adjustable from 1% to 80%. A lower set E_{SENS} increases inspiration time and a higher set E_{SENS} decreases inspiration time. The E_{SENS} setting may be utilized to limit unnecessary expiratory work and to improve patient-ventilator synchrony.

### Inspiratory Sensitivity

As discussed above, ventilation module 212 may oversee ventilation of a patient according to prescribed ventilatory settings. In one embodiment, the inspiratory sensitivity (I_{SENS} or alternatively known as trigger sensitivity) is set by a clinician or operator. According to embodiments, I_{SENS} sets the level of spontaneous effort (pressure or flow) that must be achieved by a patient to trigger or begin inspiration and to terminate exhalation. In some embodiments, the clinician or operator determines the I_{SENS} setting, which is adjustable. The I_{SENS} setting may be utilized to limit unnecessary inspiratory work and to improve patient-ventilator synchrony.

### IE Sync Breath Type

The IE Sync breath delivers inspiration and expiration during ventilation of a spontaneously breathing patient based on monitored or estimated intrapleural pressure. The term “intrapleural pressure,” as used herein, refers generally to the pressure exerted by the patient's diaphragm on the cavity in the thorax that contains the lungs, or the pleural cavity, and should further represent estimates of the pressure and/or any derivatives thereof. The use of intrapleural pressure is an effective way to determine inspiratory and expiratory patient effort. When a patient makes an effort to breathe, the patient's diaphragm will contract, and decrease the intrapleural pressure in order to draw air (or another substance) into the lungs. Because the contraction of the diaphragm is the effect of patient effort, the intrapleural pressure change is the first and a direct way to determine patient effort, as a pressure/flow change will happen subsequently. Therefore, a trigger detection application that uses intrapleural pressure is more sensitive to patient efforts than a trigger detection application that only uses pressure or flow. Accordingly, the IE Sync breath type promotes patient-ventilator synchrony. The improved synchrony provided by the IE Sync breath type minimizes patient discomfort.

The triggering described above based on intrapleural pressure as utilized by the IE Sync breath type is an additional trigger type. Accordingly, this IE Sync trigger type (or intrapleural pressure trigger type) may be an active trigger type (i.e., when utilized during the IE Sync breath type to deliver ventilation) or a background trigger type (when utilized in background).

Intrapleural pressure is estimated by the IE Sync algorithm according to any suitable method either known or discovered in the future. The intrapleural pressure estimates and their associated values can be utilized to monitor ventilation in all modes. In some embodiments, the IE Sync breath type derives intrapleural pressure readings from other data and measurements according to mathematical operations or otherwise. For example, an algorithm that estimates how the patient's intrapleural pressure is changing in real-time or quasi-real based on measured pressure and flow may be used. In one embodiment, an algorithm utilizes measured pressure, inlet flow, and outlet flow to determine intrapleural pressure. In some embodiments, the measured pressure and flow are derived from data taken by internal sensors 220 and distributed sensors 218. An example algorithm for determining intrapleural pressure is described in U.S. patent application Ser. No. 12/980,583 filed Dec. 29, 2010. Accordingly, U.S. patent application Ser. No. 12/980,583 filed on Dec. 29, 2010, is incorporated herein by reference in its entirety.

### Diaphragmatic Electromyography Adjust Breath Type

The DEA breath delivers inspiration and expiration during ventilation of a spontaneously breathing patient based on monitored neural respiratory output for the diaphragm, which is monitored with an electromyograph. The neural respiratory output, which the act of breathing depends on, is the result of a rhythmic discharge from the center of brain. The discharge is carried to the diaphragm muscles cells via the phrenic nerve causing the diaphragm muscles to contract. The contraction of diaphragm muscles causes the lungs to expand dropping pressure in the airways of lungs to provide an inflow of air into the lungs.

The neural output is the captured electrical activity of the diaphragm (Edi). The Edi is then fed to the ventilator and used by the ventilator to assist the patient's breathing. The Edi curve and its associated values can be utilized to monitor ventilation in all modes. For example, the Edi curve and its associated values may be utilized to determine respiratory drive, volume requirements, effect of a ventilation setting, and gain indications for sedation and weaning.

Because the ventilator and the diaphragm are triggered utilizing the same signal, the mechanical coupling between the ventilator and the diaphragm is almost instantaneous. The Edi signal may be utilized to trigger inspiration and expiration; therefore, the DEA breath type promotes patient-ventilator synchrony. Further, with the DEA breath type the patient's own respiratory demand is utilized to determine the level of assistance helping to provide the correct breathing assistance to the patient. Accordingly, the improved synchrony provided by the DEA breath type minimizes patient discomfort.

The triggering described above based on Edi and utilized by the DEA breath type is an additional trigger type. Accordingly, this DEA trigger type (or Edi trigger type) may be an active trigger type (i.e., when utilized during the DEA breath type to deliver ventilation) or a background trigger type (when utilized in the background).

### Ventilator Sensory Devices

The ventilatory system 200 may also include one or more distributed sensors 218 communicatively coupled to ventilator 202. Distributed sensors 218 may communicate with various components of ventilator 202, e.g., ventilation module 212, internal sensors 220, data processing module 222, asynchrony module 224, and any other suitable components and/or modules. Distributed sensors 218 may detect changes in ventilatory parameters indicative of asynchrony, for example. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Distributed sensors 218 may further include various flowmeters for detecting airflow (e.g., differential pressure pneumotachometers). For example, some flowmeters may use obstructions to create a pressure decrease corresponding to the flow across the device (e.g., differential pressure pneumotachometers) and other flowmeters may use turbines such that flow may be determined based on the rate of turbine rotation (e.g., turbine flowmeters). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors 220. Similar to distributed sensors 218, internal sensors 220 may communicate with various components of ventilator 202, e.g., ventilation module 212, internal sensors 220, data processing module 222, asynchrony module 224, and any other suitable components and/or modules. Internal sensors 220 may employ any suitable sensory or derivative technique for monitoring one or more parameters associated with the ventilation of a patient. However, the one or more internal sensors 220 may be placed in any suitable internal location, such as, within the ventilatory circuitry or within components or modules of ventilator 202. For example, sensors may be coupled to the inspiratory and/or expiratory modules for detecting changes in, for example, circuit pressure and/or flow. Specifically, internal sensors 220 may include pressure transducers and flowmeters for measuring changes in circuit pressure and airflow. Additionally or alternatively, internal sensors 220 may utilize optical or ultrasound techniques for measuring changes in ventilatory parameters. For example, a patient's expired gases may be monitored by internal sensors 220 to detect physiological changes indicative of the patient's condition and/or treatment, for example. Indeed, internal sensors 220 may employ any suitable mechanism for monitoring parameters of interest in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors, as described above, or may be indirectly monitored by derivation according to the Equation of Motion.

### Ventilatory Data

Ventilator 202 may further include a data processing module 222. As noted above, distributed sensors 218 and internal sensors 220 may collect data regarding various ventilatory parameters. Ventilator data refers to any ventilatory parameter or setting. A ventilatory parameter refers to any factor, characteristic, or measurement associated with the ventilation of a patient, whether monitored by the ventilator or by any other device. A ventilatory setting refers to any factor, characteristic, or measurement that is set by the ventilator and/or operator. Sensors may further transmit collected data to the data processing module 222 and, according to embodiments, the data processing module 222 may be configured to collect data regarding some ventilatory parameters, to derive data regarding other ventilatory parameters, and to graphically represent collected and derived data to the clinician and/or other modules of the ventilatory system 200. Some collected, derived, and/or graphically represented data may be indicative of asynchrony. For example, data regarding expiratory time, exhaled tidal volume, inspiratory time setting (T₁), etc., may be collected, derived, and/or graphically represented by data processing module 222.

### Flow Data

For example, according to embodiments, data processing module 222 may be configured to monitor inspiratory and expiratory flow. Flow may be measured by any appropriate, internal or distributed device or sensor within the ventilatory system 200. As described above, flowmeters may be employed by the ventilatory system 200 to detect circuit flow. However, any suitable device either known or developed in the future may be used for detecting airflow in the ventilatory circuit.

Data processing module 222 may be further configured to plot monitored flow data graphically via any suitable means. For example, according to embodiments, flow data may be plotted versus time (flow waveform), versus volume (flow-volume loop), or versus any other suitable parameter as may be useful to a clinician. According to embodiments, flow may be plotted such that each breath may be independently identified. Further, flow may be plotted such that inspiratory flow and expiratory flow may be independently identified, e.g., inspiratory flow may be represented in one color and expiratory flow may be represented in another color. According to additional embodiments, flow waveforms and flow-volume loops, for example, may be represented alongside additional graphical representations, e.g., representations of volume, pressure, etc., such that clinicians may substantially simultaneously visualize a variety of ventilatory parameters associated with each breath.

As may be appreciated, flow decreases as resistance increases, making it more difficult to pass gases into and out of the lungs (i.e., F=Pₜ/R). For example, when a patient is intubated, i.e., having either an endotracheal or a tracheostomy tube in place, resistance may be increased as a result of the smaller diameter of the tube over a patient's natural airway. In addition, increased resistance may be observed in patients with obstructive disorders, such as COPD, asthma, etc. Higher resistance may necessitate, inter alia, a higher inspiratory time setting (T_{I}) for delivering a prescribed pressure or volume of gases, a higher flow setting for delivering prescribed pressure or volume, a lower respiratory rate resulting in a higher expiratory time (T_{E}) for complete exhalation of gases, etc.

Specifically, changes in flow may be detected by evaluating various flow data. For example, by evaluating FV loops, as described above, an increase in resistance may be detected over a number of breaths. That is, upon comparing consecutive FV loops, the expiratory plot for each FV loop may reflect a progressive reduction in expiratory flow (i.e., a smaller FV loop), indicative of increasing resistance.

### Pressure Data

According to embodiments, data processing module 222 may be configured to monitor pressure. Pressure may be measured by any appropriate, internal or distributed device or sensor within the ventilatory system 200. For example, pressure may be monitored by proximal electromechanical transducers connected near the airway opening (e.g., on the inspiratory limb, expiratory limb, at the patient interface, etc.). Alternatively, pressure may be monitored distally, at or near the lungs and/or diaphragm of the patient.

For example, P_{Peak} and/or P_{Plat} (estimating Pₐ) may be measured proximally (e.g., at or near the airway opening) via single-point pressure measurements. According to embodiments, Pₚₗₐₜ (estimating Pₐ) may be measured during an inspiratory pause maneuver (e.g., expiratory and inspiratory valves are closed briefly at the end of inspiration for measuring the Pₚₗₐₜ at zero flow). According to other embodiments, circuit pressure may be measured during an expiratory pause maneuver (e.g., expiratory and inspiratory valves are closed briefly at the end of exhalation for measuring EEP at zero flow).

Data processing module 222 may be further configured to plot monitored pressure data graphically via any suitable means. For example, according to embodiments, pressure data may be plotted versus time (pressure waveform), versus volume (pressure-volume loop or PV loop), or versus any other suitable parameter as may be useful to a clinician. According to embodiments, pressure may be plotted such that each breath may be independently identified. Further, pressure may be plotted such that inspiratory pressure and expiratory pressure may be independently identified, e.g., inspiratory pressure may be represented in one color and expiratory pressure may be represented in another color. According to additional embodiments, pressure waveforms and PV loops, for example, may be represented alongside additional graphical representations, e.g., representations of volume, flow, etc., such that a clinician may substantially simultaneously visualize a variety of parameters associated with each breath.

According to embodiments, PV loops may provide useful clinical and diagnostic information to clinicians regarding the respiratory resistance or compliance of a patient. Specifically, upon comparing PV loops from successive breaths, an increase in resistance may be detected when successive PV loops shorten and widen over time. That is, at constant pressure, less volume is delivered to the lungs when resistance is increasing, resulting in a shorter, wider PV loop. According to alternative embodiments, a PV loop may provide a visual representation, in the area between the inspiratory plot of pressure vs. volume and the expiratory plot of pressure vs. volume, which is indicative of respiratory compliance. Further, PV loops may be compared to one another to determine whether compliance has changed. Additionally or alternatively, optimal compliance may be determined That is, optimal compliance may correspond to the dynamic compliance determined from a PV loop during a recruitment maneuver, for example.

According to additional embodiments, PV curves may be used to compare C_{S} and C_{D} over a number of breaths. For example, a first PV curve may be plotted for C_{S} (based on P_{Plat} less EEP) and a second PV curve may be plotted for C_{D} (based on P_{Peak} less EEP). Under normal conditions, C_{S} and C_{D} curves may be very similar, with the C_{D} curve mimicking the C_{S} curve but shifted to the right (i.e., plotted at higher pressure). However, in some cases the C_{D} curve may flatten out and shift to the right relative to the C_{S} curve. This graphical representation may illustrate increasing Pₜ, and thus increasing R, which may be due to mucous plugging or bronchospasm, for example. In other cases, both the C_{D} curve and the C_{S} curves may flatten out and shift to the right. This graphical representation may illustrate an increase in P_{Peak} and P_{Plat}, without an increase in Pₜ, and thus may implicate a decrease in lung compliance, which may be due to tension pneumothorax, atelectasis, pulmonary edema, pneumonia, bronchial intubation, etc.

As may be further appreciated, relationships between resistance, static compliance, dynamic compliance, and various pressure readings may give indications of patient condition. For example, when C_{S} increases, C_{D} increases and, similarly, when R increases, C_{D} increases. Additionally, as discussed previously, Pₜ represents the difference in pressure attributable to resistive forces over elastic forces. Thus, where P_{Peak} and Pₜ are increasing with constant V_{T} delivery, R is increasing (i.e., where Pₚₑₐₖ is increasing without a concomitant increase in P_{Plat}). Where Pₜ is roughly where P_{Peak} and P_{Plat} are increasing with a constant V_{T} delivery, C_{S} is increasing.

### Volume Data

According to embodiments, data processing module 222 may be configured to derive volume via any suitable means. For example, as described above, during volume ventilation, a prescribed V_{T} may be set for delivery to the patient. The actual volume delivered may be derived by monitoring the inspiratory flow over time (i.e., V=F*T). Stated differently, integration of flow over time will yield volume. According to embodiments, V_{T} is completely delivered upon reaching T_{I}. Similarly, the expiratory flow may be monitored such that expired tidal volume (V_{TE}) may be derived. That is, under ordinary conditions, upon reaching the T_{E}, the prescribed V_{T} delivered should be completely exhaled and FRC should be reached. However, under some conditions T_{E} is inadequate for complete exhalation and FRC is not reached.

Data processing module 222 may be further configured to plot derived volume data graphically via any suitable means. For example, according to embodiments, volume data may be plotted versus time (volume waveform), versus flow (flow-volume loop or FV loop), or versus any other suitable parameter as may be useful to a clinician. According to embodiments, volume may be plotted such that each breath may be independently identified. Further, volume may be plotted such that prescribed V_{T} and V_{TE} may be independently identified, e.g., prescribed V_{T} may be represented in one color and V_{TE} may be represented in another color. According to additional embodiments, volume waveforms and FV loops, for example, may be represented alongside additional graphical representations, e.g., representations of pressure, flow, etc., such that a clinician may substantially simultaneously visualize a variety of parameters associated with each breath.

### Disconnection of the Patient Ventilator Circuit

According to embodiments, data processing module 222 may be configured to determine if the ventilation tubing system 130 or patient circuit has become disconnected from the patient or the ventilator during ventilation. Data processing module 222 determines that a patient circuit is disconnected by any suitable means. In some embodiments, data processing module 222 determines that the patient circuit is disconnected by evaluating data, such as exhaled pressure and/or exhaled volume. In further embodiments, data processing module 222 determines if the patient circuit is disconnected by determining if a disconnect alarm has been executed. A disconnect alarm is executed when the ventilation tubing system is disconnected from the patient and/or the ventilator. If the disconnect alarm has been executed, then data processing module 222 determines that the patient circuit is disconnected. If the disconnect alarm has not been executed, then data processing module 222 determines that the patient circuit is connected.

### Breath Type

According to embodiments, data processing module 222 is configured to identify the breath type. In some embodiments, data processing module 222 is configured to identify the active trigger type utilized by the breath type and/or the background trigger type utilized in the background. Data processing module 222 determines the breath type and/or trigger type by any suitable systems or methods. In some embodiments, data processing module 222 determines the breath type and/or trigger type based on clinician or operator input and/or selection. In further embodiments, data processing module 222 determines the breath type and/or trigger type based on ventilator selection of the breath type and/or trigger type. For example, some breath types include VC, PC, VC+, PS, PA, IE Sync, DEA, TC, and VS.

### Asynchrony Detection

A recent study suggests that clinicians are able to detect less than one-third of patient efforts that do not result in the delivery of a breath, or missed breaths.¹ Further, this study has shown that the rate of correct detection decreases as the prevalence of missed breaths increases. Considering that missed breaths may occur in up to 80% of mechanically ventilated patients, systems and methods for better detection of asynchrony are needed. While operating a ventilator, it is desirable to detect, limit, or preferably eliminate, asynchrony. ¹Colombo, D., Cammarota, G., Alemani, M., Carenzo, L., Barra, F., Vaschetto, R., et al. (2011). Efficacy of ventilator waveforms observation in detecting patient-ventilator asynchrony. Critical Care Medicine, p. 3.

Accordingly, ventilator 202 may further include asynchrony module 224. Asynchrony is detected by the asynchrony module 224 when an ineffective effort, an auto-trigger, a late trigger, an early cycle, a late cycle, a double trigger, an inadequate flow, and/or a high tidal volume is detected. As illustrated in FIG. 2B, the asynchrony module includes an ineffective effort detection module 224a and/or an auto-trigger detection module 224c for detecting these various asynchrony conditions. In some embodiments, the detection of one type of asynchrony can cause or lead to the detection of another type of asynchrony. The asynchrony module 224 receives and analyzes data gathered by or data derived from data gathered by internal sensors 220 and/or distributed sensors 218.

### Ineffective Effort Detection

Ventilator 202 may further include an ineffective effort detection module 224a. An ineffective effort, as used herein, is a patient inspiratory effort that does not result in the delivery of a breath by the ventilator. An ineffective effort occurs when the ventilator does not detect a patient inspiratory expiratory effort. The ventilator may not detect the inspiratory because the trigger threshold is set too high and/or because the inspiratory is below the minimum trigger detection level for the trigger type. Accordingly, ineffective efforts can lead to patient discomfort, patient fatigue, and/or extended ventilation time.

The ineffective effort detection module 224a determines ineffective efforts by monitoring at least one of a pressure integral and a flow integral during exhalation for a moving window of a predetermined amount of time. In some embodiments, the moving time window is a time period of 200 ms. In other embodiments, the moving time window is a time period from 100 ms to 300 ms, such as 190 ms, 210 ms, 225 ms, 175 ms, 150 ms, or 250 ms. The moving time window progresses with the exhalation time. For example, if the moving time window is 200 ms and exhalation has lasted 300 ms, the ineffective effort detection module 224a analyzes the area under the flow and/or pressure curve during exhalation from 100 ms to the current 300 ms. In another example, if exhalation has lasted 350 ms and the moving time window is 150 ms, the ineffective effort detection module 224a analyzes the area under the flow and/or pressure curve during exhalation from 200 ms to the current 350 ms. In some embodiments, the moving windows are different amounts of time for the flow integral and the pressure integral. In alternative embodiments, the moving windows are the same amounts of time for the flow integral and the pressure integral.

In some embodiments, the ineffective effort detection module 224a detects an ineffective effort when the flow integral (∫Q_{dt}) breaches a predetermined threshold. In other embodiments, the ineffective effort detection module 224a detects an ineffective effort when the pressure integral (∫P_{dt}) breaches a predetermined threshold. In alternative embodiments, the ineffective effort detection module 224a detects an ineffective effort when the pressure integral and the flow integral breach a predetermined threshold. In some embodiments, the predetermined threshold is a pressure integral during exhalation for a 200 ms moving window of less than −0.2 cmH₂O*s. In other embodiments, the predetermined threshold is a flow integral during exhalation for a 200 ms moving window that is greater than a tidal volume derived from PBW of the patient (also referred to herein as a PBW-based tidal volume). In alternative embodiments, the predetermined threshold is a pressure integral during exhalation for a 200 ms moving window of less than −0.2 cmH₂O*s and a flow integral during exhalation for a 200 ms moving window that is greater than a PBW-based tidal volume. This list of predetermined thresholds is exemplary and is not meant to be limiting. Other suitable predetermined thresholds may be utilized by the ineffective effort detection module 224a for various time windows as would be understood by a person of skill in the art.

### Auto-Trigger Detection

Ventilator 202 may further include an auto-trigger detection module 224c. An auto-trigger, as used herein, occurs when the ventilator delivers inspiration prior to a patient's desire for an inspiration or prior to a patient's inspiratory effort. As used herein the term “prior” refers to an event that occurs more than 5 ms before a referenced event. The auto-trigger may be from some anomalous condition that is interpreted by the ventilator breath type as an inspiratory patient effort. The auto-trigger and early delivered inspiration may come before the patient has the chance to fully exhale and may cause gas-trapping in the lungs. Accordingly, auto-triggering can lead to patient discomfort.

In some embodiments, how an auto-trigger is detected by the auto-trigger detection module 224c varies based on the set breath type or ventilation mode. For example, in some embodiments, if the set breath type is a PA or TC breath type or a PS breath type with a low pressure support setting (e.g., 5 cmH₂O), the auto-trigger detection module 224c determines auto-triggers by monitoring tidal volume and respiration rate. In further embodiments, if the set ventilation mode is an AC or a SIMV mode, the auto-trigger detection module 224c determines auto-triggers by monitoring respiration rate and expiratory time.

In other embodiments, how an auto-trigger is detected by the auto-trigger detection module 224c can be utilized regardless of the set breath type or ventilation mode. In some embodiments, the auto-trigger detection module 224c determines auto-triggers by monitoring respiration rate and heart rate (HR). In further embodiments, the auto-trigger detection module 224c determines auto-triggers by monitoring expiratory time for a predetermined number of breaths.

In some embodiments, the auto-trigger detection module 224c detects an auto-trigger when: respiration rate and tidal volume breach a predetermined threshold; respiration rate and expiratory time breach a predetermined threshold; respiration rate breaches a predetermined threshold; and/or expiratory time breaches a predetermined threshold.

In some embodiments, the predetermined threshold is a tidal volume of less than 1 mL/kg and a respiration rate greater than a respiration rate derived from a PBW of the patient (also referred to herein as PBW-based RR), which both occur for a predetermined duration. The predetermined duration may be a predetermined amount of time or a predetermined number of breaths. In some embodiments the predetermined duration is 10 seconds. In other embodiments, the predetermined duration is 4 second, 5 seconds, 6 seconds, 7 seconds, 8 seconds, 9 seconds, 11 seconds, 12 seconds, 3 breaths, 4 breaths, 5 breaths, 6 breaths, 7 breaths, 8 breaths, 9 breaths, 10 breaths, or 11 breaths.

In other embodiments, the predetermined threshold is a respiration rate that is greater than a PBW-based RR, a respiration rate that is greater than a set respiration rate, and an expiratory time that is constant (little or no variability), which all occur for a predetermined duration. This predetermined duration may be a predetermined amount of time or a predetermined number of breaths. In some embodiments, this predetermined duration is 10 seconds. In other embodiments, the predetermined duration is 4 second, 5 seconds, 6 seconds, 7 seconds, 8 seconds, 9 seconds, 11 seconds, 12 seconds, 3 breaths, 4 breaths, 5 breaths, 6 breaths, 7 breaths, 8 breaths, 9 breaths, 10 breaths, or 11 breaths.

In further embodiments, the predetermined threshold is a respiration rate that is greater than a PBW-based RR and the presence of cardiogenic noise, which both occur for a predetermined duration. In some embodiments, cardiogenic noise is detected when the respiration rate is congruent with or substantially equivalent to a patient HR) for a predetermined duration. In some embodiments, the respiration rate is considered to be congruent with or substantially equivalent with the heart rate when the respiration rate is greater than 0.9*HR. This predetermined duration may also be a predetermined amount of time or a predetermined number of breaths. In some embodiments, this predetermined duration is 5 seconds. In other embodiments, the predetermined duration is 3 seconds, 4 seconds, 6 seconds, 7 seconds, 8 seconds, 9 seconds, 11 seconds, 12 seconds, 1 breath, 2 breaths, 3 breaths, 4 breaths, 5 breaths, 6 breaths, 7 breaths, 8 breaths, 9 breaths, 10 breaths, or 11 breaths.

In additional embodiments, the predetermined threshold is an expiratory time that equals the minimum expiratory time for a predetermined duration. This predetermined duration may also be a predetermined amount of time or a predetermined number of breaths. In some embodiments, this predetermined duration is 3 breaths. In other embodiments, the predetermined duration is 1 second, 2 seconds, 3 seconds, 4 seconds, 5 seconds, 6 seconds, 7 seconds, 8 seconds, 9 seconds, 11 seconds, 12 seconds, 1 breath, 2 breaths, 4 breaths, 5 breaths, 6 breaths, 7 breaths, 8 breaths, 9 breaths, 10 breaths, or 11 breaths. In some embodiments, the minimum expiratory time is 200 ms. As understood by a person of skill in the art the minimum expiratory time may vary based on the sex, age, weight, PBW, height, and/or disease state of the patient.

### Smart-Prompt Generation

Ventilator 202 may further include a prompt such as a smart prompt module 226. As may be appreciated, multiple ventilatory parameters may be monitored and evaluated in order to detect an implication of asynchrony. In addition, when asynchrony is implicated, many clinicians may not be aware of adjustments to ventilatory parameters that may reduce or eliminate asynchrony. As such, upon detection of asynchrony, the smart prompt module 226 may be configured to notify the clinician that asynchrony is implicated and/or to provide recommendations to the clinician for mitigating asynchrony. For example, smart prompt module 226 may be configured to notify the clinician by displaying a smart prompt on display module 204 and/or within a window of the GUI. According to additional embodiments, the smart prompt is communicated to and/or displayed on a remote monitoring system communicatively coupled to ventilatory system 200. According to alternative embodiments, the smart prompt is any audio and/or visual notification. Alternatively, in an automated embodiment, the smart prompt module 226 communicates with a ventilator control system so that the recommendation may be automatically implemented to mitigate asynchrony.

In order to accomplish the various aspects of the notification and/or recommendation message display, the smart prompt module 226 may communicate with various other components and/or modules. For instance, smart prompt module 226 may be in communication with data processing module 222, asynchrony module 224, or any other suitable module or component of the ventilatory system 200. That is, smart prompt module 226 may receive an indication that asynchrony has been implicated by any suitable means. In addition, smart prompt module 226 may receive information regarding one or more parameters that implicated the presence of asynchrony and information regarding the patient's ventilatory settings and treatment. Further, according to some embodiments, the smart prompt module 226 may have access to a patient's diagnostic information (e.g., regarding whether the patient has ARDS, COPD, asthma, emphysema, or any other disease, disorder, or condition).

Smart prompt module 226 may further comprise additional modules for making notifications and/or recommendations to a clinician regarding the presence of asynchrony. For example, according to embodiments, smart prompt module 226 includes a notification module 228 and a recommendation module 230. For instance, smart prompts may be provided according to a hierarchical structure such that a notification message and/or a recommendation message may be initially presented in summarized form and, upon clinician selection, an additional detailed notification and/or recommendation message may be displayed. According to alternative embodiments, a notification message is initially presented and, upon clinician selection, a recommendation message may be displayed. Alternatively or additionally, the notification message may be simultaneously displayed with the recommendation message in any suitable format or configuration.

Specifically, according to embodiments, the notification message alerts the clinician as to the detection of a patient condition, a change in patient condition, or an effectiveness of ventilatory treatment. For example, the notification message may alert the clinician that asynchrony has been detected and the type of asynchrony detected. The type of asynchrony detected may include an ineffective effort and/or an auto-trigger. The notification message may further alert the clinician regarding the particular ventilatory parameter(s) that implicated asynchrony (e.g., low trigger sensitivity resulted in an ineffective effort, etc.)

Additionally, according to embodiments, the recommendation message provides various suggestions to the clinician for addressing a detected condition. That is, if asynchrony has been detected, the recommendation message may suggest that the clinician consider changing to a different breath type. According to additional embodiments, the recommendation message may be based on particular ventilatory parameter(s) (e.g., respiration rate, expiratory time, tidal volume, etc.) that implicated asynchrony. Additionally or alternatively, the recommendation message may be based on current ventilatory settings (e.g., breath type, patient interface type, and etc.) such that suggestions are directed to a particular patient's treatment. Additionally or alternatively, the recommendation message may be based on secondary conditions (e.g., elevated resistance, leak detection, and etc.) such that suggestions are directed to a particular patient's treatment. Additionally or alternatively, the recommendation message may be based on a diagnosis and/or other patient attributes. Further still, the recommendation message may include a primary recommendation message and a secondary recommendation message.

As described above, smart prompt module 226 may also be configured with notification module 228 and recommendation module 230. The notification module 228 may be in communication with data processing module 222, asynchrony module 224, or any other suitable module to receive an indication that asynchrony has been detected. Notification module 228 may be responsible for generating a notification message via any suitable means. For example, the notification message may be provided as a tab, banner, dialog box, or other similar type of display. Further, the notification messages may be provided along a border of the graphical user interface, near an alarm display or bar, or in any other suitable location. A shape and size of the notification message may further be optimized for easy viewing with minimal interference to other ventilatory displays. The notification message may be further configured with a combination of icons and text such that the clinician may readily identify the message as a notification message.

The recommendation module 230 may be responsible for generating one or more recommendation messages via any suitable means. The one or more recommendation messages may provide suggestions and information regarding addressing a detected condition and may be accessible from the notification message. For example, the one or more recommendation messages may identify the parameters that implicated the detected condition, may provide suggestions for adjusting one or more ventilatory parameters to address the detected condition, may provide suggestions for checking ventilatory equipment or patient position, or may provide other helpful information. Specifically, the one or more recommendation messages may provide suggestions and information regarding asynchrony.

According to embodiments, based on the particular parameters that implicated asynchrony, the recommendation module 230 provides suggestions for addressing asynchrony. In some embodiments, the recommendation module 230 provides suggestions for addressing asynchrony based on the type of asynchrony detected. That is, if auto-trigger is implicated, the one or more recommendation messages may include suggestions or recommendations for the following:
adjust trigger sensitivity to a less sensitive setting;
enable leak compensation;
change a trigger type;
check patient circuit for condensate;
check seal of patient interface; and
any other suitable suggestion or recommendation.
   Alternatively, if an ineffective effort is implicated, the one or more recommendation messages may include suggestions or recommendations for the following:
shorten an inspiration time by increasing a peak flow setting or by changing a waveform shape to square;
lower a tidal volume setting;
check for causes of increased resistance;
check for a need to suction or deliver a bronchodilator;
reduce an inspiratory sensitivity setting or change to a more sensitive trigger type;
increase an expiratory sensitivity setting or increase a rise time setting;
lower a pressure support setting;
correct a leak;
enable leak compensation;
increase expiratory sensitivity setting;
lower inspiratory pressure setting;
increase expiratory sensitivity setting
change to an IE sync trigger type; and
any other suitable suggestion or recommendation.

According to still other embodiments, the recommendation message includes a primary message and a secondary message. That is, a primary message may provide notification of the condition detected and/or suggestions that are specifically targeted to the detected condition based on the particular parameters that implicated the condition. Alternatively, the primary message may provide suggestions that may provide a higher likelihood of mitigating the detected condition. The secondary message may provide more general suggestions and/or information that may aid the clinician in further addressing and/or mitigating the detected condition. For example, the primary message may provide a specific suggestion for adjusting a particular parameter to mitigate the detected condition (e.g., consider adjusting trigger sensitivity). Alternatively, the secondary message may provide general suggestions for addressing the detected condition.

Additionally or alternatively, the one or more recommendation messages may also be based on a secondary condition or current ventilator settings for the patient and/or the type of asynchrony detected. In some embodiments, the secondary conditions include the detection of Auto PEEP, detection of elevated resistance, detection of a long inspiration time, detection of a high tidal volume, and/or leak detection. In some embodiments, the ventilator settings include breath type, ventilation mode, and/or patient interface type. For example, when auto-triggering is implicated, the one or more recommendation messages may vary based on whether or not leak is detected and/or based on the type of patient interface utilized. For example, the recommendation may change based on whether the patient circuit is invasive (e.g., endotracheal tube) or non-invasive (e.g., facial mask). In another example, when an ineffective effort is implicated, the one or more recommendation messages may vary based on whether or not an elevated resistance is detected and/or whether or not a leak is detected. In some embodiments, an elevated resistance for an adult is detected when the determined or measured patient resistance is greater than 10 cm H₂O/liter/s. In other embodiments, an elevated resistance is detected when the measured or determined resistance of the patient is greater than the resistance derived from the PBW of the patient (also referred to as PBW-based resistance) by at least 10%. Other secondary conditions may be detected when a predetermined threshold is breached that varies based on the sex, age, height, weight, IBW, PBW, and disease state of the person as would be known by a person of skill in the art. In another embodiment, if asynchrony was implicated during a PC breath type by detecting an auto-trigger, where the patient's current ventilator settings includes an invasive patient interface (e.g., an endotracheal tube), then the one or more recommendation messages may suggest that the clinician check the patient circuit for condensate. Further, in this example, a secondary recommendation message may suggest to adjust trigger sensitivity to a more sensitive setting.

The detection of a an auto-trigger as described above informs the ventilator that the breath type is not responding correctly to patient efforts based on the patient's respiration rate and/or expiratory time. Accordingly, in some embodiments, this information is utilized in a notification message and/or recommendation message to the clinician (e.g., decrease trigger sensitivity because respiration rate is high). In an alternative embodiment, the ventilator automatically adjusts trigger sensitivity based on the detected auto-trigger.

Table 1 below lists various examples of primary and secondary recommendations for a detected auto-trigger or a detected ineffective effort based on ventilation modes, breath types, ventilator settings, secondary conditions, and/or detection method.

TABLE 1
Recommendation messages based on a ventilation mode, breath type, ventilator
setting, secondary condition, detection method, and/or type of asynchrony.

| | | | Secondary | | | |
|---|---|---|---|---|---|---|
| Vent. | | | Condition | | | |
| Mode/ | | | and/or | | Primary | Secondary |
| Breath | Detection | Type of | Ventilator | Notif. | Recommendation | Recommendation |
| Type | Method | Asynch. | Settings | Message | Message | Message |
| Any | RR > PBW- | Auto- | No leak | Auto- | Consider | OR, consider |
| breath | based RR and | trigger | detected | trigger | decreasing trigger | changing trigger |
| type | RR ≈ HR for at | | | detected | sensitivity | type |
| | least 5 | | | | | |
| Any | expiratory time = | Auto- | No leak | Auto- | Consider | OR, consider |
| breath | minimum | trigger | detected | trigger | decreasing trigger | changing trigger |
| type | expiratory time | | | detected | sensitivity | type |
| | (e.g., 200 ms) | | | | | |
| | for more than 3 | | | | | |
| | consecutive | | | | | |
| | breaths | | | | | |
| PA or | no/low volume | Auto- | No leak | Auto- | Consider | OR, consider |
| TC, or | (e.g., <1 | trigger | detected | trigger | decreasing trigger | changing trigger |
| PS that | mL/kg) and | | | detected | sensitivity | type |
| provides | RR > PBW- | | | | | |
| less | based RR | | | | | |
| than 5 | for >10 seconds | | | | | |
| cm H₂O | | | | | | |
| AC or | RR > set RR | Auto- | No leak | Auto- | Consider | OR, consider |
| SIMV | and RR > | trigger | detected | trigger | decreasing trigger | changing trigger |
| | PBW-based | | | detected | sensitivity | type |
| | RR and | | | | | |
| | expiratory time | | | | | |
| | is constant (no | | | | | |
| | variability) | | | | | |
| | for >10 seconds | | | | | |
| Any | RR > PBW- | Auto- | Leak present | Auto- | Consider enabling | N/A |
| breath | based RR and | trigger | | trigger | leak compensation | |
| type | RR ≈ HR for at | | | detected | | |
| | least 5 seconds | | | | | |
| Any | expiratory time = | Auto- | Leak present | Auto- | Consider enabling | N/A |
| breath | minimum | trigger | | trigger | leak compensation | |
| type | expiratory time | | | detected | | |
| | (e.g., 200 ms) | | | | | |
| | for more than 3 | | | | | |
| | consecutive | | | | | |
| | breaths | | | | | |
| PA or | no/low volume | Auto- | Leak present | Auto- | Consider enabling | N/A |
| TC, or | (e.g., <1 | trigger | | trigger | leak compensation | |
| PS that | mL/kg) and | | | detected | | |
| provides | RR > PBW- | | | | | |
| less | based RR | | | | | |
| than 5 | for >10 seconds | | | | | |
| cm H₂O | | | | | | |
| AC or | RR > set RR | Auto- | Leak present | Auto- | Consider enabling | N/A |
| SIMV | and RR > | trigger | | trigger | leak compensation | |
| | PBW-based | | | detected | | |
| | RR and | | | | | |
| | expiratory time | | | | | |
| | is constant (no | | | | | |
| | variability) | | | | | |
| | for >10 seconds | | | | | |
| Any | RR > PBW- | Auto- | No leak | Auto- | Consider | OR, consider |
| breath | based RR and | trigger | detected | trigger | decreasing trigger | checking patient |
| type | RR ≈ HR for at | | | detected | sensitivity | circuit for |
| | least 5 seconds | | | | | condensate |
| Any | expiratory time = | Auto- | No leak | Auto- | Consider | OR, consider |
| breath | minimum | trigger | detected | trigger | decreasing trigger | checking patient |
| type | expiratory time | | | detected | sensitivity | circuit for |
| | (e.g., 200 ms) | | | | | condensate |
| | for more than 3 | | | | | |
| | consecutive | | | | | |
| | breaths | | | | | |
| PA or | no/low volume | Auto- | No leak | Auto- | Consider | OR, consider |
| TC, or | (e.g., <1 | trigger | detected | trigger | decreasing trigger | checking patient |
| PS that | mL/kg) and | | | detected | sensitivity | circuit for |
| provides | RR > PBW- | | | | | condensate |
| less | based RR | | | | | |
| than 5 | for >10 seconds | | | | | |
| cm H₂O | | | | | | |
| AC or | RR > set RR | Auto- | No leak | Auto- | Consider | OR, consider |
| SIMV | and RR > | trigger | detected | trigger | decreasing trigger | checking patient |
| | PBW-based | | | detected | sensitivity | circuit for |
| | RR and | | | | | condensate |
| | expiratory time | | | | | |
| | is constant (no | | | | | |
| | variability) | | | | | |
| | for >10 seconds | | | | | |
| Any | RR > PBW- | Auto- | Invasive | Auto- | Consider enabling | OR, consider |
| breath | based RR and | trigger | circuit type, | trigger | leak | changing trigger |
| type | RR ≈ HR for at | | Leak detected | detected | compensation, OR | type, OR consider |
| | least 5 seconds | | | | consider checking | increasing trigger |
| | | | | | the patient circuit | sensitivity |
| | | | | | for condensate | |
| Any | expiratory time = | Auto- | Invasive | Auto- | Consider enabling | OR, consider |
| breath | minimum | trigger | circuit type, | trigger | leak | changing trigger |
| type | expiratory time | | Leak detected | detected | compensation, OR | type, OR consider |
| | (e.g., 200 ms) | | | | consider checking | increasing trigger |
| | for more than 3 | | | | the patient circuit | sensitivity |
| | consecutive | | | | for condensate | |
| | breaths | | | | | |
| PA or | no/low volume | Auto- | Invasive | Auto- | Consider enabling | OR, consider |
| TC, or | (e.g., <1 | trigger | circuit type, | trigger | leak | changing trigger |
| PS that | mL/kg) and | | Leak detected | detected | compensation, OR | type, OR consider |
| provides | RR > PBW- | | | | consider checking | increasing trigger |
| less | based RR | | | | the patient circuit | sensitivity |
| than 5 | for >10 seconds | | | | for condensate | |
| cm H₂O | | | | | | |
| AC or | RR > set RR | Auto- | Invasive | Auto- | Consider enabling | OR, consider |
| SIMV | and RR> | trigger | circuit type, | trigger | leak | changing trigger |
| | PBW-based | | Leak detected | detected | compensation, OR | type, OR consider |
| | RR and | | | | consider checking | increasing trigger |
| | expiratory time | | | | the patient circuit | sensitivity |
| | is constant (no | | | | for condensate | |
| | variability) | | | | | |
| | for >10 seconds | | | | | |
| Any | RR > PBW- | Auto- | Non-invasive | Auto- | Consider enabling | OR, consider |
| breath | based RR and | trigger | circuit type, | trigger | leak | changing trigger |
| type | RR ≈ HR for at | | Leak detected | detected | compensation, OR | type, OR consider |
| | least 5 seconds | | | | consider checking | increasing trigger |
| | | | | | the seal of the | sensitivity, OR |
| | | | | | interface device | consider checking |
| | | | | | | the patient circuit |
| | | | | | | for condensate |
| Any | expiratory time = | Auto- | Non-invasive | Auto- | Consider enabling | OR, consider |
| breath | minimum | trigger | circuit type, | trigger | leak | changing trigger |
| type | expiratory time | | Leak detected | detected | compensation, OR | type, OR consider |
| | (e.g., 200 ms) | | | | consider checking | increasing trigger |
| | for more than 3 | | | | the seal of the | sensitivity, OR |
| | consecutive | | | | interface device | consider checking |
| | breaths | | | | | the patient circuit |
| | | | | | | for condensate |
| PA or | no/low volume | Auto- | Non-invasive | Auto- | Consider enabling | OR, consider |
| TC, or | (e.g., <1 | trigger | circuit type, | trigger | leak | changing trigger |
| PS that | mL/kg) and | | Leak detected | detected | compensation, OR | type, OR consider |
| provides | RR > PBW- | | | | consider checking | increasing trigger |
| less | based RR | | | | the seal of the | sensitivity, OR |
| than 5 | for >10 seconds | | | | interface device | consider checking |
| cm H₂O | | | | | | the patient circuit |
| | | | | | | for condensate |
| AC or | RR > set RR | Auto- | Non-invasive | Auto- | Consider enabling | OR, consider |
| SIMV | and RR > | trigger | circuit type, | trigger | leak | changing trigger |
| | PBW-based | | Leak detected | detected | compensation, OR | type, OR consider |
| | RR and | | | | consider checking | increasing trigger |
| | expiratory time | | | | the seal of the | sensitivity, OR |
| | is constant (no | | | | interface device | consider checking |
| | variability) | | | | | the patient circuit |
| | for >10 seconds | | | | | for condensate |
| VC | ∫ P_{dt} during | Ineffective | Auto PEEP | Ineffective | Consider | OR, consider |
| | exhalation for | effort | detected, no | effort | shortening T_{I} by | shortening T_{I} by |
| | a 200 ms | | elevated | detected | increasing peak | changing |
| | moving | | resistance | | flow setting | waveform shape to |
| | window < −0.2 | | detected, and | | | square |
| | cm H₂O*s | | no high tidal | | | |
| | and/or ∫ Q_{dt} | | volume | | | |
| | during | | detected | | | |
| | exhalation for | | | | | |
| | a 200 ms | | | | | |
| | moving | | | | | |
| | window > | | | | | |
| | PBW-based | | | | | |
| | volume | | | | | |
| VC | ∫ P_{dt} during | Ineffective | Auto PEEP | Ineffective | Consider lowering | N/A |
| | exhalation for | effort | detected, no | effort | the tidal volume | |
| | a 200 ms | | elevated | detected | setting | |
| | moving | | resistance | | | |
| | window < −0.2 | | detected, and | | | |
| | cm H₂O*s | | high tidal | | | |
| | and/or ∫ Q_{dt} | | volume | | | |
| | during | | detected | | | |
| | exhalation for | | | | | |
| | a 200 ms | | | | | |
| | moving | | | | | |
| | window > | | | | | |
| | PBW-based | | | | | |
| | volume | | | | | |
| VC | ∫ P_{dt} during | Ineffective | Auto PEEP | Ineffective | Consider checking | OR consider |
| | exhalation for | effort | detected, | effort | causes for | checking for a |
| | a 200 ms | | elevated | detected | increased R | need to suction or |
| | moving | | resistance | | | consider |
| | window < −0.2 | | detected, no | | | delivering a |
| | cm H₂O*s | | long T_{I} | | | bronchodilator |
| | and/or ∫ Q_{dt} | | detected, and | | | |
| | during | | no high tidal | | | |
| | exhalation for | | volume | | | |
| | a 200 ms | | detected | | | |
| | moving | | | | | |
| | window > | | | | | |
| | PBW-based | | | | | |
| | volume | | | | | |
| VC | ∫ P_{dt} during | Ineffective | No Auto | Ineffective | Consider reducing | OR consider |
| | exhalation for | effort | PEEP | effort | the trigger | changing to a |
| | a 200 ms | | detected, no | detected | sensitivity setting | more sensitive |
| | moving | | elevated | | | trigger type |
| | window < −0.2 | | resistance | | | |
| | cm H₂O*s | | detected, no | | | |
| | and/or ∫ Q_{dt} | | long T_{I} | | | |
| | during | | detected, no | | | |
| | exhalation for | | high tidal | | | |
| | a 200 ms | | volume | | | |
| | moving | | detected, and | | | |
| | window > | | no leak | | | |
| | PBW-based | | detected | | | |
| | volume | | | | | |
| PS | ∫ P_{dt} during | Ineffective | No Auto | Ineffective | Consider reducing | OR consider |
| | exhalation for | effort | PEEP | effort | the trigger | changing to a |
| | a 200 ms | | detected, no | detected | sensitivity setting | more sensitive |
| | moving | | elevated | | | trigger type |
| | window < −0.2 | | resistance | | | |
| | cm H₂O*s | | detected, no | | | |
| | and/or ∫ Q_{dt} | | long T_{I} | | | |
| | during | | detected, no | | | |
| | exhalation for | | high tidal | | | |
| | a 200 ms | | volume | | | |
| | moving | | detected, and | | | |
| | window > | | no leak | | | |
| | PBW-based | | detected | | | |
| | volume | | | | | |
| PS | ∫ P_{dt} during | Ineffective | Auto PEEP | Ineffective | Consider checking | OR consider |
| | exhalation for | effort | detected, | effort | causes for | checking for a |
| | a 200 ms | | elevated | detected | increased R | need to suction or |
| | moving | | resistance | | | consider |
| | window < −0.2 | | detected, no | | | delivering a |
| | cm H₂O*s | | high tidal | | | bronchodilator |
| | and/or ∫ Q_{dt} | | volume | | | |
| | during | | detected, and | | | |
| | exhalation for | | no leak | | | |
| | a 200 ms | | detected | | | |
| | moving | | | | | |
| | window > | | | | | |
| | PBW-based | | | | | |
| | volume | | | | | |
| PS | ∫ P_{dt} during | Ineffective | Auto PEEP | Ineffective | Consider | OR, consider |
| | exhalation for | effort | detected, no | effort | increasing E_{SENS} | increasing rise |
| | a 200 ms | | elevated | detected | setting | time setting |
| | moving | | resistance | | | |
| | window < −0.2 | | detected, a | | | |
| | cm H₂O*s | | long T_{I} | | | |
| | and/or ∫ Q_{dt} | | detected, no | | | |
| | during | | high tidal | | | |
| | exhalation for | | volume | | | |
| | a 200 ms | | detected, and | | | |
| | moving | | no leak | | | |
| | window > | | detected | | | |
| | PBW-based | | | | | |
| | volume | | | | | |
| PS | ∫ P_{dt} during | Ineffective | No elevated | Ineffective | Consider | N/A |
| | exhalation for | effort | resistance | effort | decreasing the | |
| | a 200 ms | | detected, a | detected | pressure support | |
| | moving | | high tidal | | setting | |
| | window < −0.2 | | volume | | | |
| | cm H₂O*s | | detected, and | | | |
| | and/or ∫ Q_{dt} | | no leak | | | |
| | during | | detected | | | |
| | exhalation for | | | | | |
| | a 200 ms | | | | | |
| | moving | | | | | |
| | window > | | | | | |
| | PBW-based | | | | | |
| | volume | | | | | |
| PS | ∫ P_{dt} during | Ineffective | No Auto | Ineffective | Consider | OR consider |
| | exhalation for | effort | PEEP | effort | correcting leak, or | increasing the |
| | a 200 ms | | detected, no | detected | enabling leak | E_{SENS} setting |
| | moving | | elevated | | comp, | |
| | window < −0.2 | | resistance | | | |
| | cm H₂O*s | | detected, a | | | |
| | and/or ∫ Q_{dt} | | long T_{I} | | | |
| | during | | detected, no | | | |
| | exhalation for | | high tidal | | | |
| | a 200 ms | | volume | | | |
| | moving | | detected, and | | | |
| | window > | | leak detected | | | |
| | PBW-based | | | | | |
| | volume | | | | | |
| PC | ∫ P_{dt} during | Ineffective | Auto PEEP | Ineffective | Consider lowering | N/A |
| | exhalation for | effort | detected, no | effort | inspiratory | |
| | a 200 ms | | elevated | detected | pressure setting | |
| | moving | | resistance | | | |
| | window < −0.2 | | detected, and | | | |
| | cm H₂O*s | | a high tidal | | | |
| | and/or ∫ Q_{dt} | | volume | | | |
| | during | | detected | | | |
| | exhalation for | | | | | |
| | a 200 ms | | | | | |
| | moving | | | | | |
| | window > | | | | | |
| | PBW-based | | | | | |
| | volume | | | | | |
| VC+ | ∫ P_{dt} during | Ineffective | No Auto | Ineffective | Consider | OR, consider |
| | exhalation for | effort | PEEP and | effort | increasing trigger | changing the |
| | a 200 ms | | inspiration | detected | sensitivity | trigger type |
| | moving | | time is in | | | |
| | window < −0.2 | | normal range | | | |
| | cm H₂O*s | | based PBW | | | |
| | and/or ∫ Q_{dt} | | | | | |
| | during | | | | | |
| | exhalation for | | | | | |
| | a 200 ms | | | | | |
| | moving | | | | | |
| | window > | | | | | |
| | PBW-based | | | | | |
| | volume | | | | | |
| VC+ | ∫ P_{dt} during | Ineffective | Tidal Volume | Ineffective | Consider | N/A |
| | exhalation for | effort | too large for | effort | decreasing tidal | |
| | a 200 ms | | PBW | detected | volume | |
| | moving | | | | | |
| | window < −0.2 | | | | | |
| | cm H₂O*s | | | | | |
| | and/or ∫ Q_{dt} | | | | | |
| | during | | | | | |
| | exhalation for | | | | | |
| | a 200 ms | | | | | |
| | moving | | | | | |
| | window > | | | | | |
| | PBW-based | | | | | |
| | volume | | | | | |
| VC+ | ∫ P_{dt} during | Ineffective | Auto PEEP, | Ineffective | Consider | N/A |
| | exhalation for | effort | respiration | effort | decreasing | |
| | a 200 ms | | rate, and | detected | inspiration time | |
| | moving | | inspiration | | | |
| | window < −0.2 | | time too long | | | |
| | cm H₂O*s | | for PBW | | | |
| | and/or ∫ Q_{dt} | | | | | |
| | during | | | | | |
| | exhalation for | | | | | |
| | a 200 ms | | | | | |
| | moving | | | | | |
| | window > | | | | | |
| | PBW-based | | | | | |
| | volume | | | | | |
| VC+ | ∫ P_{dt} during | Ineffective | Auto PEEP | Ineffective | Consider | OR consider |
| | exhalation for | effort | detected | effort | increasing PEEP | decreasing |
| | a 200 ms | | | detected | | respiration rate if |
| | moving | | | | | exhalation volume |
| | window < −0.2 | | | | | is high or consider |
| | cm H₂O*s | | | | | decreasing |
| | and/or ∫ Q_{dt} | | | | | inspiration time |
| | during | | | | | |
| | exhalation for | | | | | |
| | a 200 ms | | | | | |
| | moving | | | | | |
| | window > | | | | | |
| | PBW-based | | | | | |
| | volume | | | | | |
| VS | ∫ P_{dt} during | Ineffective | No Auto | Ineffective | Consider | OR, consider |
| | exhalation for | effort | PEEP | effort | increasing trigger | changing the |
| | a 200 ms | | detected, no | detected | sensitivity | trigger type |
| | moving | | elevated | | | |
| | window < −0.2 | | resistance | | | |
| | cm H₂O*s | | detected, no | | | |
| | and/or ∫ Q_{dt} | | long T_{I} | | | |
| | during | | detected, no | | | |
| | exhalation for | | high tidal | | | |
| | a 200 ms | | volume | | | |
| | moving | | detected, and | | | |
| | window > | | no leak | | | |
| | PBW-based | | detected | | | |
| | volume | | | | | |
| VS | ∫ P_{dt} during | Ineffective | Auto PEEP | Ineffective | Consider checking | OR consider |
| | exhalation for | effort | detected, | effort | causes for | checking for a |
| | a 200 ms | | elevated | detected | increased R | need to suction or |
| | moving | | resistance | | | consider |
| | window < −0.2 | | detected, no | | | delivering a |
| | cm H₂O*s | | high tidal | | | bronchodilator |
| | and/or ∫ Q_{dt} | | volume | | | |
| | during | | detected, and | | | |
| | exhalation for | | no leak | | | |
| | a 200 ms | | detected | | | |
| | moving | | | | | |
| | window > | | | | | |
| | PBW-based | | | | | |
| | volume | | | | | |
| VS | ∫ P_{dt} during | Ineffective | Auto PEEP | Ineffective | Consider | N/A |
| | exhalation for | effort | detected, no | effort | increasing E_{SENS} | |
| | a 200 ms | | elevated | detected | setting | |
| | moving | | resistance | | | |
| | window < −0.2 | | detected, a | | | |
| | cm H₂O*s | | long T_{I} | | | |
| | and/or J∫ | | detected, no | | | |
| | exhalation for | | high tidal | | | |
| | a 200 ms | | volume | | | |
| | moving | | detected, and | | | |
| | window > | | no leak | | | |
| | PBW-based | | detected | | | |
| | volume | | | | | |
| VS | ∫ P_{dt} during | Ineffective | No elevated | Ineffective | Consider lowering | N/A |
| | exhalation for | effort | resistance | effort | tidal volume | |
| | a 200 ms | | detected, a | detected | setting | |
| | moving | | high tidal | | | |
| | window < −0.2 | | volume | | | |
| | cm H₂O*s | | detected, and | | | |
| | and/or ∫ Q_{dt} | | no leak | | | |
| | during | | detected | | | |
| | exhalation for | | | | | |
| | a 200 ms | | | | | |
| | moving | | | | | |
| | window > | | | | | |
| | PBW-based | | | | | |
| | volume | | | | | |
| VS | ∫ P_{dt} during | Ineffective | Auto PEEP | Ineffective | Consider | OR consider |
| | exhalation for | effort | detected, no | effort | correcting leak, or | increasing the |
| | a 200 ms | | elevated | detected | enabling leak | E_{SENS} setting |
| | moving | | resistance | | comp, | |
| | window < −0.2 | | detected, a | | | |
| | cm H₂O*s | | long T_{I} | | | |
| | and/or ∫ Q_{dt} | | detected, no | | | |
| | during | | high tidal | | | |
| | exhalation for | | volume | | | |
| | a 200 ms | | detected, and | | | |
| | moving | | leak detected | | | |
| | window > | | | | | |
| | PBW-based | | | | | |
| | volume | | | | | |
| PA | ∫ P_{dt} during | Ineffective | Auto PEEP | Ineffective | Consider checking | OR consider |
| | exhalation for | effort | detected, | effort | causes for | checking for a |
| | a 200 ms | | elevated | detected | increased R | need to suction or |
| | moving | | resistance | | | consider |
| | window < −0.2 | | detected, no | | | delivering a |
| | cm H₂O*s | | long T_{I} | | | bronchodilator |
| | and/or ∫ Q_{dt} | | detected, no | | | |
| | during | | high tidal | | | |
| | exhalation for | | volume | | | |
| | a 200 ms | | detected, and | | | |
| | moving | | no leak | | | |
| | window > | | detected | | | |
| | PBW-based | | | | | |
| | volume | | | | | |
| PA | ∫ P_{dt} during | Ineffective | Auto PEEP | Ineffective | Consider | OR consider |
| | exhalation for | effort | detected, no | effort | correcting leak, or | increasing the |
| | a 200 ms | | elevated | detected | enabling leak | E_{SENS} setting |
| | moving | | resistance | | comp, | |
| | window < −0.2 | | detected, a | | | |
| | cm H₂O*s | | long T_{I} | | | |
| | and/or ∫ Q_{dt} | | detected, no | | | |
| | during | | high tidal | | | |
| | exhalation for | | volume | | | |
| | a 200 ms | | detected, and | | | |
| | moving | | leak detected | | | |
| | window > | | | | | |
| | PBW-based | | | | | |
| | volume | | | | | |

As noted above, according to embodiments, the notification message is associated with a primary prompt and the one or more recommendation messages may be associated with a secondary prompt. That is, a primary prompt may provide an alert that asynchrony has been detected and may further provide one or more potential causes for asynchrony. Alternatively, an alert may be separately provided, indicating that asynchrony was detected, and the primary prompt may provide the one or more potential causes for asynchrony. According to additional or alternative embodiments, the secondary prompt provides the one or more recommendations and/or information that may aid the clinician in further addressing and/or mitigating the detected condition. For example, the secondary prompt may recommend addressing asynchrony by adjusting an alternative parameter, by switching the breath type, and/or etc. Smart prompt module 226 may also be configured such that smart prompts (including alerts, primary prompts, and/or secondary prompts) may be displayed in a partially transparent window or format. The transparency may allow for notification and/or recommendation messages to be displayed such that normal ventilator GUI and respiratory data may be visualized behind the messages. This feature may be particularly useful for displaying detailed messages. As described previously, notification and/or recommendation messages may be displayed in areas of the display screen that are either blank or that cause minimal distraction from the respiratory data and other graphical representations provided by the GUI. However, upon selective expansion of a message, respiratory data and graphs may be at least partially obscured. As a result, translucent display may provide the detailed message such that it is partially transparent. Thus, graphical and other data may be visible behind the detailed alarm message.

Additionally, notification and/or recommendation messages may provide immediate access to the display and/or settings screens associated with the detected condition. For example, an associated parameter settings screen may be accessed from a notification and/or a recommendation message via a hyperlink such that the clinician may address the detected condition as necessary. An associated parameter display screen may also be accessed such that the clinician may view clinical data associated with the detected condition in the form of charts, graphs, or otherwise. That is, according to embodiments, the clinician accesses the ventilatory data that implicated the detected condition for verification purposes. For example, when asynchrony has been implicated, depending on the particular ventilatory parameters that implicated asynchrony, the clinician may be able to access ventilatory settings for addressing asynchrony (e.g., a settings screen for adjusting trigger sensitivity, tidal volume, pressure support level, etc.) and/or to view associated ventilatory parameters that implicated asynchrony (e.g., a graphics screen displaying historical flow waveforms, current respiration rate, and/or waveforms illustrating the asynchrony such as an ineffective effort or an auto-trigger).

According to embodiments, upon viewing the notification and/or recommendation messages, upon addressing the detected condition by adjusting one or more ventilatory settings or otherwise, or upon manual selection, the notification and/or recommendation messages are cleared from the graphical user interface. According to some embodiments, smart prompt module 226 clears the one or more messages from the graphical user interface if a setting is changed on the ventilator, such as a selected breath type. In further embodiments, smart prompt module 226 clears the one or more messages from the graphical user interface if a ventilator setting change was performed by the operator and a threshold was not breached for a predetermined amount of time or number of breaths. In further embodiments, smart prompt module 226 clears the one or more messages from the graphical user interface if the threshold breach does not occur again during a predetermined amount of time or breaths. In some embodiments, the smart prompt module 226 clears the one or more messages from the graphical user interface upon user selection.

Asynchrony Detection during Ventilation of a Patient

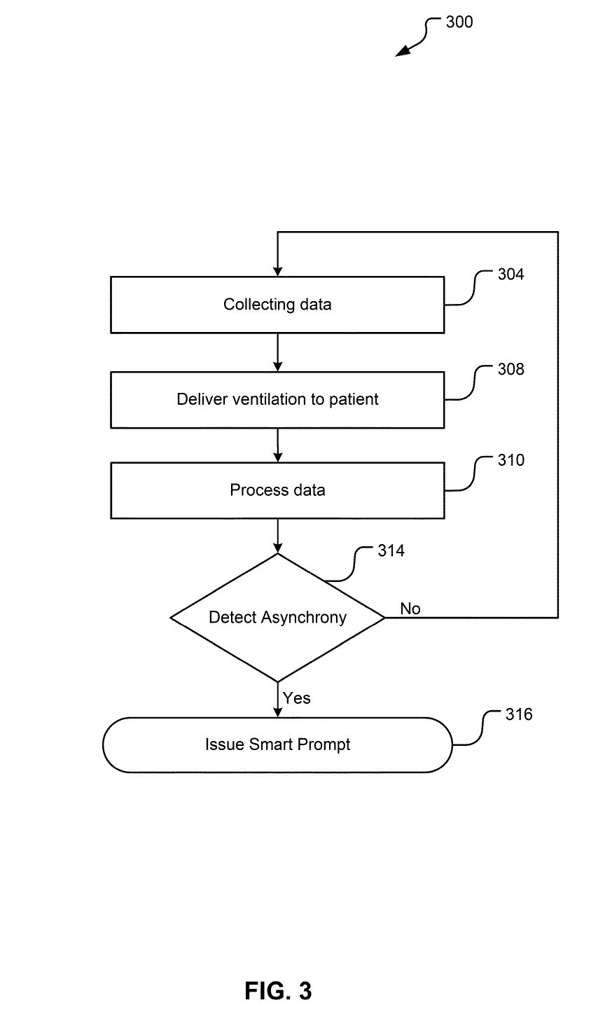

FIG. 3 is a flow chart illustrating an embodiment of a method 300 for detecting an implication of asynchrony.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

The illustrated embodiment of the method 300 depicts a method for detecting asynchrony during ventilation of a patient. Method 300 begins with collecting data operation 304. Collecting data operation 304 may include receiving data regarding one or more ventilatory settings associated with ventilation of a patient. For example, the ventilator may be configured to provide ventilation to a patient. As such, the ventilatory settings and/or input received may include a prescribed V_{T}, set flow (or peak flow), predicted or ideal body weight (PBW or IBW), E_{SENS}, trigger sensitivity, PEEP, etc. Collecting data operation 304 may include receiving data from sensors regarding one or more ventilatory parameters or receiving derived data from a processor. As discussed above, a ventilatory parameter refers to any factor, characteristic, or measurement associated with the ventilation of a patient, whether monitored by the ventilator or by any other device. The collected data may be transmitted by sensors. For example, data regarding flow rate, circuit pressure, flow pattern, expiratory time (T_{E}), etc., may be collected from the sensors, operator interface, and/or processor.

At deliver ventilation operation 308, the ventilator provides ventilation to a patient, as described above. That is, according to embodiments, the ventilator provides ventilation based on the set breath type. For example, during a VC breath type in the mixed mode, the ventilator provides ventilation based on a prescribed V_{T}. In this example, the ventilator may deliver gases to the patient at a set flow at a set RR. When prescribed V_{T} has been delivered, the ventilator may initiate the expiratory phase unless the ventilator detects a patient trigger or cycle.

While ventilation is being delivered, the ventilator may conduct various data processing operations. For example, at data processing operation 310, the ventilator collects and/or derives various ventilatory parameter data associated with ventilation of the patient. For example, as described above, the ventilator may collect data regarding parameters including T_{E}, V_{T}, T_{E} flow, pressure, respiration rate, etc. Additionally, the ventilator may derive various ventilatory parameter data based on the collected data, e.g., PBW-based T_{I}, PBW-based respiration rate, PBW-based tidal volume, respiratory resistance, respiration rate, pressure integral during exhalation for a moving window of a predetermined amount of time, flow integral during exhalation for a moving window of a predetermined amount of time, respiratory compliance, detected patient triggers, detected patient cycles, etc. As described previously, measurements for respiratory resistance and/or compliance may be trended continuously for a patient because ventilatory data may be obtained without sedating the patient or otherwise. Additionally, the ventilator may generate various graphical representations of the collected and/or derived ventilatory parameter data, e.g., flow waveforms, pressure waveforms, pressure-volume loops, flow-volume loops, etc. Additionally, in some embodiments, at data processing operation 310, the ventilator collects and/or derives and/or identifies current ventilator settings (e.g., patient interface type, breath type, ventilation mode, etc.).

According to some embodiments, at detect asynchrony operation 314 the ventilator determines whether asynchrony is implicated by evaluating expiratory time, respiratory rate, tidal volume, pressure integral during exhalation for a moving time window, flow integral during exhalation for a moving time window, etc. and comparing the evaluated parameters to one or more predetermined thresholds. In some embodiments, in order to prevent unnecessary alarms, notifications, and/or recommendations, thresholds and conditions are utilized by the detect asynchrony operation 314 to determine when asynchrony has occurred with sufficient duration to warrant notification of the operator. For example, in some embodiments, asynchrony that occurs in one breath in isolation from any other breath with asynchrony will not be considered enough to warrant an occurrence of asynchrony by detect asynchrony operation 314.

In some embodiments, at detect asynchrony operation 314 the ventilator may determine whether asynchrony is implicated based on a predetermined duration of occurrence. For example, in one embodiment, the ventilator at detect asynchrony operation 314 determines that an auto-trigger is implicated when:
a tidal volume is less than 1 mL/kg and a respiration rate is greater than a respiration rate derived from a PBW of the patient (also referred to herein as PBW-based RR) for a predetermined duration, such as 10 seconds;
a respiration rate is greater than a PBW-based RR and a set respiration rate and an expiratory time is constant (little or no variability) for a predetermined duration, such as 10 seconds;
a respiration rate is greater than a PBW-based RR and cardiogenic noise is present (e.g., the respiration rate is congruent with or substantially equivalent to a patient HR) for a predetermined duration, such as 5 seconds; and
an expiratory time equals the minimum expiratory time for a predetermined duration, such as 3 breaths.
   For example, in another embodiment, the ventilator at detect asynchrony operation 314 determines that an ineffective effort is implicated when one or more of the following conditions are met:
when a pressure integral during exhalation for a moving time window is less than −0.2 cmH₂O*s; and
when a flow integral during exhalation for a moving time window is greater than a PBW-based tidal volume.

If asynchrony is implicated, detect asynchrony operation 314 may proceed to issue smart prompt operation 316. If asynchrony is not implicated, the detect asynchrony operation 314 may return to collecting data operation 304. However, in some embodiments, the ventilator continuously performs the collecting data operation 304, the deliver ventilation operation 308, data processing operation 310, and detect asynchrony operation 314.

The thresholds listed above are just one example list of possible conditions that could be used to indicate asynchrony in the detect asynchrony operation 314. Any suitable list of conditions for determining the occurrence of asynchrony may be utilized by the detect asynchrony operation 314. As may be appreciated, the ventilator may determine whether asynchrony is implicated at detect asynchrony operation 314 via any suitable means. Indeed, any of the above described ventilatory parameters may be evaluated according to various thresholds for detecting asynchrony. Further, the disclosure regarding specific ventilatory parameters as they may implicate asynchrony is not intended to be limiting. In fact, any suitable ventilatory parameter may be monitored and evaluated for detecting asynchrony within the spirit of the present disclosure. As such, if asynchrony is implicated via any suitable means, the detect asynchrony operation 314 may proceed to issue smart prompt operation 316.

At issue smart prompt operation 316, the ventilator may alert the clinician via any suitable means that asynchrony has been implicated. For example, according to embodiments, the ventilator may display a smart prompt including a notification message and/or a recommendation message regarding the detection and/or cause of asynchrony on the GUI. According to alternative embodiments, the ventilator may communicate the smart prompt, including the notification message and/or the recommendation message, to a remote monitoring system communicatively coupled to the ventilator. According to alternative embodiments, the issued smart prompt is any visual and/or audio notification.

According to embodiments, the notification message may alert the clinician that asynchrony has been detected and, optionally, may provide information regarding the type of asynchrony and/or any ventilatory parameter(s) that implicated asynchrony. According to additional embodiments, the recommendation message may provide one or more suggestions for mitigating asynchrony. According to further embodiments, the one or more suggestions may be based on the patient's particular ventilatory settings (e.g. breath type, patient interface type etc.) and/or secondary condition (e.g., elevated resistance present, leak present, and etc.). According to some embodiments, the clinician may access one or more parameter settings and/or display screens from the smart prompt via a hyperlink or otherwise for addressing asynchrony. According to additional or alternative embodiments, a clinician may remotely access one or more parameter and/or display screens from the smart prompt via a hyperlink or otherwise for remotely addressing asynchrony.

### Smart Prompt Generation Regarding Asynchrony Detection

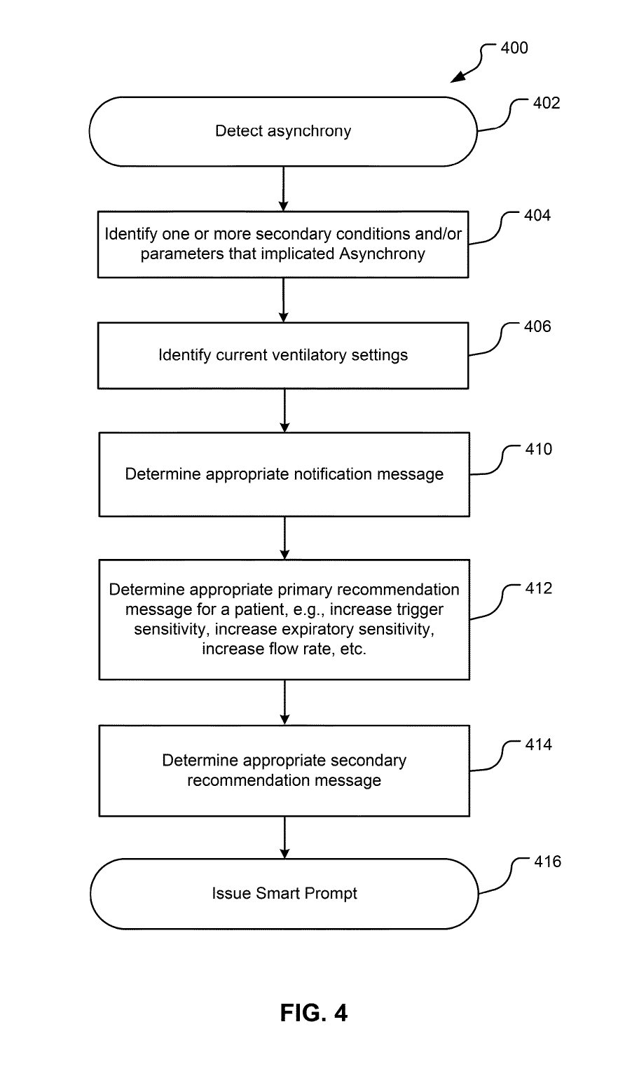

FIG. 4 is a flow chart illustrating an embodiment of a method 400 for issuing a smart prompt upon detecting an implication of asynchrony.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

The illustrated embodiment of the method 400 depicts a method for issuing a smart prompt upon detecting asynchrony during ventilation of a patient. Method 400 begins with detect operation 402, wherein the ventilator detects that asynchrony, such as an ineffective effort and auto-trigger, is implicated as described above in method 300.

At identify ventilatory parameters operation 404, the ventilator may identify one or more ventilatory parameters that implicated asynchrony and/or secondary patient conditions. For example, at operation 404, the ventilator may identify if the patient has an elevated resistance, if the patient exhibits a long inspiratory time, if the patient suffers from auto PEEP, if the patient has a high tidal volume, and/or if a leak is detected. The thresholds for determining these secondary conditions, as known by a person of skill in the art, may vary based on a patient's sex, age, height, weight, PBW, IBW, and/or disease condition. For example, as discussed above, an elevated resistance may be present in an adult when the determined or measured patient resistance is greater than 10 cm H₂O/liter/s. The secondary conditions listed above are just one example list of possible conditions that could be used to determine the appropriate recommendation message in operations 412 and/or 414. Any suitable list of conditions for determining the appropriate recommendation message may be utilized by the operation 412 and/or 414. As may be appreciated, the ventilator may also use information regarding ventilatory parameters that implicated asynchrony in determining an appropriate notification and/or recommendation message of the smart prompt. Based on the parameters that implicated the asynchrony, the ventilator during the parameters operation 404 may further identify the type of asynchrony detected, such as ineffective effort and/or auto-trigger.

At identify settings operation 406, the ventilator may identify one or more current ventilatory settings associated with the ventilatory treatment of the patient. For example, current ventilatory settings may have been received upon initiating ventilation for the patient and may have been determined by the clinician or otherwise (e.g., breath type, patient interface type, PBW or IBW, disease conditions, etc.). For instance, current ventilatory settings associated with ventilation for a patient may include, set respiration rate, patient interface type, and etc. As may be appreciated, the ventilator may use information regarding current ventilatory settings in determining an appropriate notification and/or recommendation message of the smart prompt.

At determine operation 410, the ventilator may determine an appropriate notification message. For example, the appropriate notification message may alert the clinician that asynchrony has been implicated and, optionally, may provide information regarding the type of asynchrony and the ventilatory parameter(s) that implicated asynchrony. For example, the appropriate notification may alert the clinician that asynchrony was the result of an ineffective effort detected by an analysis of the flow and/or pressure integral during exhalation for a predetermined moving time window. In another example, the appropriate notification may alert the clinician that asynchrony was the result of an auto-trigger. For example, if asynchrony was detected because of an auto-trigger, the ventilator may offer one or more notification messages that may include: “Consider adjusting trigger sensitivity to a less sensitive setting” or “consider checking the patient circuit for condensate.” In alternative embodiments, measured parameters (including secondary conditions), such as mean airway pressure, detected patient efforts, airway flow, cardiogenic noise, resistance level, and etc. may be utilized in and/or by the notification message.

At determine operation 412, the ventilator may determine an appropriate primary recommendation message. The appropriate primary recommendation message may provide one or more specific suggestions for mitigating asynchrony. According to some embodiments, in determining the appropriate primary recommendation message, the ventilator may take into consideration the one or more monitored ventilatory parameters that implicated asynchrony and the type of asynchrony detected.

According to other embodiments, in determining an appropriate primary recommendation message the ventilator may take into consideration one or more of the patient's ventilatory settings and/or secondary conditions. For example, if the breath type is volume-control (VC), if the resistance is elevated, and if ineffective effort is the type of asynchrony detected, the ventilator may offer one or more recommendation messages that may include: “Consider checking for causes of increased resistance” or “Consider checking for a need to suction or consider delivering a bronchodilator.” In another example, if the breath type is proportional assist (PA), if the patient circuit is non-invasive, and if the type of asynchrony detected is an auto-trigger, the ventilator may offer one or more recommendation messages that may include: “Consider checking the seal of the patient interface.” Any of the primary recommendations as discussed above and as displayed in Table 1 above for any breath type may be utilized by method 400.

In some embodiments, at determine operation 414, the ventilator also determines an appropriate secondary recommendation message. The secondary recommendation message may provide one or more general suggestions for mitigating asynchrony. For example, the secondary recommendation message may include: “Consider changing trigger type.” The secondary recommendation message may provide additional recommendations for mitigating asynchrony. In further embodiments, the appropriate secondary recommendation message may take into consideration the patient's current ventilatory settings and/or secondary conditions. That is, during a VC breath type, for a detected ineffective effort and a detected elevated resistance, the ventilator may suggest checking causes for increase resistance. As known by a person of skill in the art any notification, message, and/or recommendation disclosed herein may suitable for use as a primary and/or secondary recommendation message.

At issue smart prompt operation 416, a smart prompt is issued. A smart prompt is issued when the ventilator alerts the clinician via any suitable means that asynchrony has been implicated. For example, according to embodiments, a smart prompt may include an appropriate notification message and an appropriate recommendation message regarding the presence of asynchrony. Additionally or alternatively, the smart prompt may include an appropriate notification message, an appropriate primary recommendation message, and an appropriate secondary recommendation message. The smart prompt may be displayed via any suitable means, e.g., on the ventilator GUI and/or at a remote monitoring station, such that the clinician is alerted as to the potential presence of asynchrony and offered additional information and/or recommendations for mitigating asynchrony, as described herein.

In some embodiments, a ventilatory system for issuing a smart prompt when asynchrony is implicated during ventilation of a patient is disclosed. The ventilatory system includes: means for collecting data associated with ventilatory parameters; means for processing the collected ventilatory parameter data, wherein the step of processing the collected ventilatory parameter data comprises deriving ventilatory parameter data from the collected ventilatory parameter data, wherein the processed ventilator parameter data includes at least one of respiration rate and expiratory time; means for determining that an auto-trigger is implicated upon detecting that the processed ventilatory parameter data breaches a received predetermined threshold; and means for issuing a smart prompt when the ineffective effort is implicated.

In some embodiments, a ventilatory system for issuing a smart prompt when asynchrony is implicated during ventilation of a patient is disclosed. The ventilatory system includes: means for detecting an auto-trigger based on at least one of a monitored respiration rate and an expiratory time, means for identifying the current ventilator settings and secondary conditions, means for determining the appropriate notification message, means for determining the appropriate primary recommendation message and the appropriate secondary recommendation for the patient based on the identified ventilator settings and secondary conditions, and means for issuing the notification message, the primary recommendation message and the secondary recommendation message.

In some embodiments, a ventilatory system for issuing a smart prompt when asynchrony is implicated during ventilation of a patient is disclosed. The ventilatory system includes: means for collecting data associated with ventilatory parameters; means for processing the collected ventilatory parameter data, wherein the step of processing the collected ventilatory parameter data comprises deriving ventilatory parameter data from the collected ventilatory parameter data, wherein the processed ventilator parameter data includes at least one of a pressure integral during exhalation for a moving time window and a flow integral during exhalation for a moving time window; means for determining that an ineffective effort is implicated upon detecting that the processed ventilatory parameter data breaches a received predetermined threshold; and means for issuing a smart prompt when the ineffective effort is implicated.

In some embodiments, a ventilatory system for issuing a smart prompt when asynchrony is implicated during ventilation of a patient is disclosed. The ventilatory system includes: means for detecting an ineffective based on at least one of a pressure integral during exhalation for a moving time window and a flow integral during exhalation for a moving time window, means for identifying the current ventilator settings and secondary conditions, means for determining the appropriate notification message, means for determining the appropriate primary recommendation message and the appropriate secondary recommendation for the patient based on the identified ventilator settings and secondary conditions, and means for issuing the notification message, the primary recommendation message and the secondary recommendation message.

In further embodiments, the means for the medical ventilator are illustrated in FIGS. 1 and 2 and are described in the above descriptions of FIGS. 1 and 2. However, the means described above for FIGS. 1 and 2 and illustrated in FIGS. 1 and 2 are but one example only and are not meant to be limiting.

In some embodiments, a graphical user interface for displaying one or more prompts corresponding to a detected condition, a ventilator configured with a computer having a user interface including the graphical user interface for accepting commands and for displaying information is disclosed. The graphical user interface includes at least one window and one or more elements within the at least one window comprising at least one prompt element for communicating information regarding a detected auto-trigger based on a monitored respiration rate and/or expiratory time. The at least one prompt element further comprises at least one of a notification message and one or more recommendation messages. The notification message comprises one or more alerts associated with a detected auto-trigger. The one or more recommendation messages comprise one or more recommendations for mitigating the detected auto-trigger. The one or more recommendations comprise one or more of:
a recommendation to adjust trigger sensitivity to a less sensitive setting;
a recommendation to enable leak compensation;
a recommendation to change a trigger type;
a recommendation to check a patient circuit for condensate; and
a recommendation to check a seal of a patient interface.
   In some embodiments, the one or more recommendations are also based on an identified ventilator setting and/or a secondary condition.

In additional embodiments, a graphical user interface for displaying one or more prompts corresponding to a detected condition, a ventilator configured with a computer having a user interface including the graphical user interface for accepting commands and for displaying information is disclosed. The graphical user interface includes at least one window and one or more elements within the at least one window comprising at least one prompt element for communicating information regarding a detected ineffective effort based on a monitored a pressure integral during exhalation for a moving time window and/or a flow integral during exhalation for a moving time window. The at least one prompt element further comprises at least one of a notification message and one or more recommendation messages. The notification message comprises one or more alerts associated with a detected ineffective effort. The one or more recommendation messages comprise one or more recommendations for mitigating the detected ineffective effort. The one or more recommendations comprise one or more of:
a recommendation to shorten an inspiration time by increasing a peak flow setting or by changing a waveform shape to square;
a recommendation to lower a tidal volume setting;
a recommendation to check for causes of increased resistance;
a recommendation to check for a need to suction or consider a delivering a bronchodilator;
a recommendation to reduce an inspiratory sensitivity setting or to change to a more sensitive trigger type;
a recommendation to increase an expiratory sensitivity setting or to increase a rise time setting;
a recommendation to lower a pressure support setting;
a recommendation to correct a leak, to enable leak compensation, or to increase an expiratory sensitivity setting;
a recommendation to lower an inspiratory pressure setting; and
a recommendation to increase an expiratory sensitivity setting.
   In some embodiments, the one or more recommendations are also based on an identified ventilator setting and/or a secondary condition.

### Ventilator GUI Display of Initial Smart Prompt

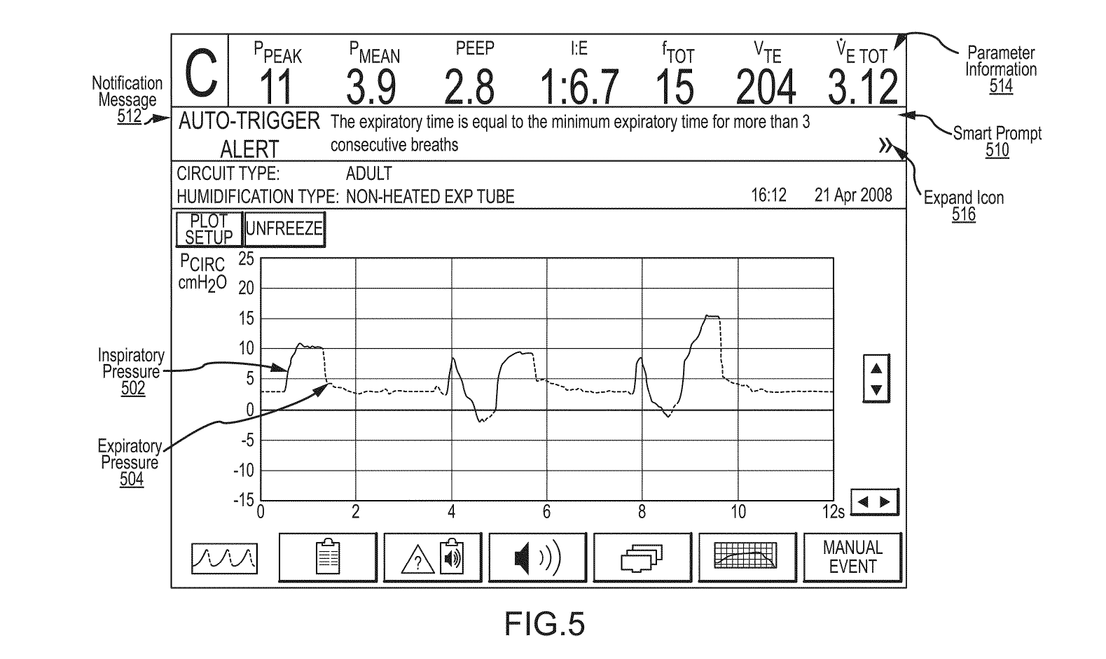

FIG. 5 is an illustration of an embodiment of a graphical user interface 500 displaying a smart prompt having a notification message 512.

Graphical user interface 500 may display various monitored and/or derived data to the clinician during ventilation of a patient. In addition, graphical user interface 500 may display various messages to the clinician (e.g., alarm messages, etc.). Specifically, graphical user interface 500 may display a smart prompt as described herein.

According to embodiments, the ventilator may monitor and evaluate various ventilatory parameters based on one or more predetermined thresholds to detect asynchrony. As illustrated, a pressure waveform may be generated and displayed by the ventilator on graphical user interface 500. As further illustrated, the pressure waveform may be displayed such that pressure during inspiration 502 is represented in a different color (e.g., green) than pressure during expiration 504 (e.g., yellow).

Upon a determination that asynchrony is implicated, the graphical user interface 500 may display a smart prompt, e.g., smart prompt 510. According to embodiments, smart prompt 510 may be displayed in any suitable location such that a clinician may be alerted regarding a detected patient condition, but while allowing other ventilatory displays and data to be visualized substantially simultaneously. As illustrated, smart prompt 510 is presented as a bar or banner across an upper region of the graphical user interface 500. However, as previously noted, smart prompt 510 may be displayed as a tab, icon, button, banner, bar, or any other suitable shape or form. Further, smart prompt 510 may be displayed in any suitable location within the graphical user interface 500. For example, smart prompt 510 may be located along any border region of the graphical user interface 500 (e.g., top, bottom, or side borders) (not shown), across an upper region (shown), or in any other suitable location. Further, as described herein, smart prompt 510 may be partially transparent (not shown) such that ventilatory displays and data may be at least partially visible behind smart prompt 510.

Specifically, smart prompt 510 may alert the clinician that asynchrony has been detected, for example by notification message 512. As described herein, notification message 512 may alert the clinician that asynchrony is implicated via any suitable means, e.g., “Ineffective Effort Alert,” “Asynchrony Alert,” “Asynchrony Detected,” “Asynchrony Implicated,” “Auto-Trigger Alert,” “Auto-trigger Detected,” “Auto-Trigger Implicated,”, “Ineffective Effort Implicated,” “Ineffective Effort Detected,” or etc. Smart prompt 510 may further include information regarding ventilatory parameters that implicated asynchrony. For example, if asynchrony was detected based on a respiration rate or an expiratory time, then this information may be displayed by the notification message 512 (e.g., “Auto-Trigger Alert—Respiration Rate is too high” or “Auto-Trigger Detected—Expiratory time is equal to the minimum expiratory time for more than 3 consecutive breaths”). According to the illustrated embodiment, parameter information 514 is provided along with the notification message 512 in a banner. According to alternative embodiments, in addition to the notification message 512 and the parameter information 514, one or more recommendation messages may be provided in an initial smart prompt banner (not shown). According to other embodiments, rather than providing information regarding ventilatory parameters that implicated asynchrony in the initial smart prompt, this information may be provided within an expanded portion (not shown) of smart prompt 510.

According to embodiments, smart prompt 510 may be expanded to provide additional information and/or recommendations to the clinician regarding a detected patient condition. For example, an expand icon 516 may be provided within a suitable area of the smart prompt 510. According to embodiments, upon selection of the expand icon 516 via any suitable means, the clinician may optionally expand the smart prompt 510 to acquire additional information and/or recommendations for mitigating the detected patient condition. According to further embodiments, smart prompt 510 may include links to additional settings and/or display screens of the graphical user interface 500 such that the clinician may easily and quickly mitigate and/or verify the detected condition.

As may be appreciated, the disclosed data, graphics, and smart prompt illustrated in graphical user interface 500 may be arranged in any suitable order or configuration such that information and alerts may be communicated to the clinician in an efficient and orderly manner. The disclosed data, graphics, and smart prompt are not to be understood as an exclusive array, as any number of similar suitable elements may be displayed for the clinician within the spirit of the present disclosure. Further, the disclosed data, graphics, and smart prompt are not to be understood as a necessary array, as any number of the disclosed elements may be appropriately replaced by other suitable elements without departing from the spirit of the present disclosure. The illustrated embodiment of the graphical user interface 500 is provided as an example only, including potentially useful information and alerts that may be provided to the clinician to facilitate communication of detected set asynchrony in an orderly and informative way, as described herein.

### Ventilator GUI Display of Expanded Smart Prompt

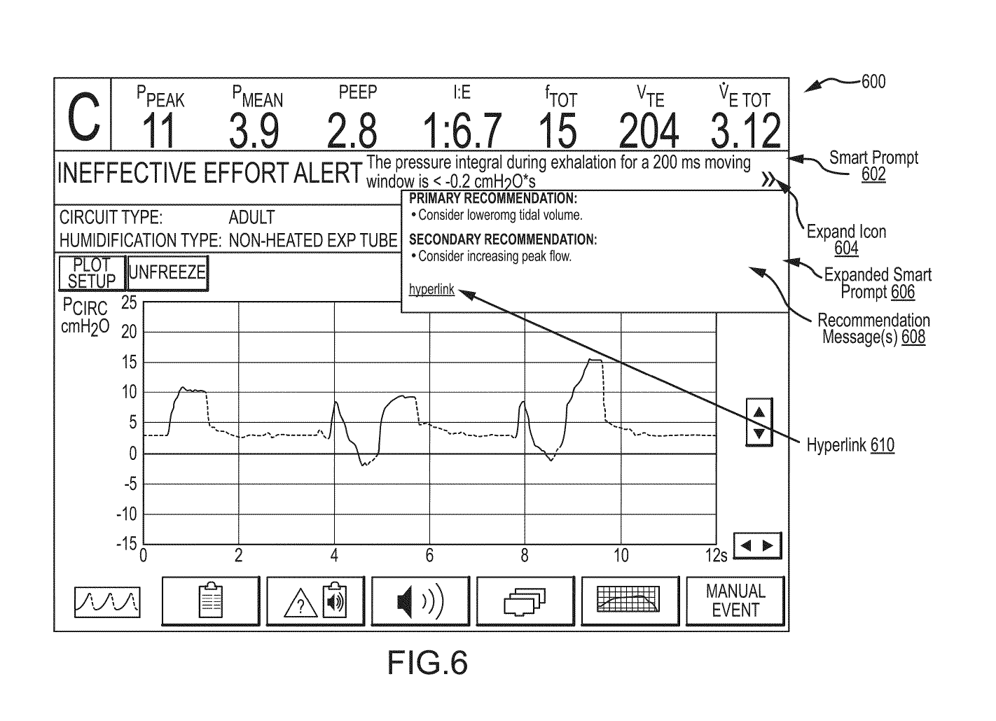

FIG. 6 is an illustration of an embodiment of a graphical user interface 600 displaying an expanded smart prompt 606 having a notification message and one or more recommendation messages 608.

Graphical user interface 600 may display various monitored and/or derived data to the clinician during ventilation of a patient. In addition, graphical user interface 600 may display an expanded smart prompt 606 including one or more recommendation messages 608 as described herein.

According to embodiments, as described above, an expand icon 604 may be provided within a suitable area of smart prompt 602. Upon selection of the expand icon 604, the clinician may optionally expand smart prompt 602 to acquire additional information and/or recommendations for mitigating the detected patient condition. For example, expanded smart prompt 606 may be provided upon selection of expand icon 604. As described above for smart prompt 510, expanded smart prompt 606 may be displayed as a tab, icon, button, banner, bar, or any other suitable shape or form. Further, expanded smart prompt 606 may be displayed in any suitable location within the graphical user interface 600. For example, expanded smart prompt 606 may be displayed below (as shown) smart prompt 602, to a side of smart prompt 602, or otherwise logically associated with smart prompt 602. According to other embodiments, an initial smart prompt may be hidden upon displaying expanded smart prompt 606. Expanded smart prompt 606 may also be partially transparent such that ventilatory displays and data may be at least partially visible behind expanded smart prompt 606.

According to embodiments, expanded smart prompt 606 may comprise additional information and/or one or more recommendation messages 608 regarding detected asynchrony. For example, the one or more recommendation messages 608 may include a primary recommendation message and a secondary recommendation message. The primary recommendation message may provide one or more specific suggestions for mitigating asynchrony. For example, the primary recommendation message may include: “Consider lowering tidal volume.” The secondary recommendation message may provide one or more general suggestions for mitigating asynchrony. For example, the secondary recommendation message may include: “Consider changing breath type,” or “Consider changing trigger type.”

According to embodiments, expanded smart prompt 606 may also include one or more hyperlinks 610, which may provide immediate access to the display and/or settings screens associated with detected asynchrony. For example, associated parameter settings screens may be accessed from expanded smart prompt 606 via hyperlinks 610 such that the clinician may address detected asynchrony by adjusting one or more parameter settings as necessary. Alternatively, associated parameter display screens may be accessed such that the clinician may view clinical data associated with asynchrony in the form of charts, graphs, or otherwise. That is, according to embodiments, the clinician may access the ventilatory data that implicated asynchrony for verification purposes. For example, when asynchrony has been implicated, depending on the particular ventilatory parameters that implicated asynchrony, the clinician may be able to access associated parameter settings screens for addressing asynchrony (e.g., settings screens for adjusting respiration rate, changing trigger type, for changing breath type, etc.). Additionally or alternatively, the clinician may be able to access and/or view display screens associated with the ventilatory parameters that implicated asynchrony (e.g., a graphics screen displaying historical flow waveforms, volume waveforms, and/or pressure waveforms that give rise to implications of asynchrony).

As may be appreciated, the disclosed smart prompt and recommendation messages 608 illustrated in graphical user interface 600 may be arranged in any suitable order or configuration such that information and alerts may be communicated to the clinician in an efficient and orderly manner. Indeed, the illustrated embodiment of the graphical user interface 600 is provided as an example only, including potentially useful information and recommendations that may be provided to the clinician to facilitate communication of suggestions for mitigating detected asynchrony in an orderly and informative way, as described herein.

Unless otherwise indicated, all numbers expressing measurements, dimensions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. Further, unless otherwise stated, the term “about” shall expressly include “exactly,” consistent with the discussions regarding ranges and numerical data. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 4 percent to about 7 percent” should be interpreted to include not only the explicitly recited values of about 4 percent to about 7 percent, but also include individual values and sub-ranges within the indicated range. Thus, included in this numerical range are individual values such as 4.5, 5.25 and 6 and sub-ranges such as from 4-5, from 5-7, and from 5.5-6.5, etc. This same principle applies to ranges reciting only one numerical value. Furthermore, such an interpretation should apply regardless of the breadth of the range or the characteristics being described.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims.

What is claimed is:

1. A ventilator-implemented method for detecting asynchrony during ventilation of a patient, the method comprising:
collecting data associated with ventilatory parameters;
processing the collected ventilatory parameter data, wherein processing the collected ventilatory parameter data comprises deriving ventilatory parameter data from the collected ventilatory parameter data, wherein the processed ventilator parameter data includes at least one of a respiration rate and an expiratory time;
determining that an auto-trigger is implicated upon detecting that the processed ventilator parameter data breaches a received predetermined threshold; and
issuing a smart prompt when the auto-trigger is implicated.

2. The method of claim 1, further comprising:
identifying that the patient is being ventilated with one of a pressure assist breath type, a tube compensation breath type, or a pressure support breath type that provides less than 5 cm of H₂O to the patient,
wherein the processed ventilator parameter data is a tidal volume and the respiration rate, and
wherein the received predetermined threshold is a determined tidal volume of less than 1 mL/kg for more than 10 seconds and a determined respiration rate of less than a derived respiration rate based on a predicted body weight of the patient for more than 10 seconds.

3. The method of claim 1, further comprising:
identifying that the patient is being ventilated with one of an assist control or a synchronized intermittent mandatory ventilation (SIMV) mode,
wherein the processed ventilator parameter data is the respiration rate and the expiratory time, and
wherein the received predetermined threshold is a determined respiration rate of greater than a set respiration rate and greater than a derived respiration rate based on a predicted body weight of the patient for more than 10 seconds and a determined expiratory time that does not vary for more than 10 seconds.

4. The method of claim 1, wherein the processed ventilator parameter data is the respiration rate and a heart rate, and
wherein the received predetermined threshold is a determined respiration rate of greater than a derived respiration rate from a predicted body weight of the patient for at least 5 seconds and that is substantially equivalent to the heart rate of the patient for at least 5 seconds.

5. The method of claim 4, wherein the determined respiration rate is substantially equivalent to the heart rate when the determined respiration rate is greater than 0.9 times the heart rate.

6. The method of claim 1, wherein the processed ventilator parameter data is the expiratory time, and
wherein the received predetermined threshold is a determined expiratory time of greater than a minimum expiratory time for more than three consecutive breaths.

7. The method of claim 1, wherein the collected ventilatory parameter data comprises:
at least one of airway flow and airway pressure.

8. The method of claim 1, further comprising:
determining an appropriate recommendation message for the issued smart prompt based at least in part on identifying at least one secondary condition,
wherein the at least one secondary condition includes determining if a leak is detected, and
wherein the appropriate recommendation message includes one of:
a recommendation to adjust trigger sensitivity to a less sensitive setting;
a recommendation to enable leak compensation;
a recommendation to change a trigger type;
a recommendation to check a patient circuit for condensate; and
a recommendation to check a seal of a patient interface.

9. The method of claim 1, further comprising:
determining an appropriate recommendation message for the issued smart prompt based at least in part on identifying one or more ventilatory settings associated with a ventilatory treatment of the patient,
wherein the one or more ventilatory settings include a patient interface type, and
wherein the appropriate recommendation message includes one of:
a recommendation to adjust trigger sensitivity to a less sensitive setting;
a recommendation to enable leak compensation;
a recommendation to change a trigger type;
a recommendation to check a patient circuit for condensate; and
a recommendation to check a seal of a patient interface.

10. A ventilatory system for issuing a smart prompt when asynchrony is implicated during ventilation of a patient, comprising:
at least one processor; and
at least one memory, communicatively coupled to the at least one processor and containing instructions that are executed by the at least one processor, the instructions comprising:
detecting that an auto-trigger is implicated for the patient based on at least one of a respiration rate and an expiratory time;
determining an appropriate notification message;
determining an appropriate recommendation message; and
issuing at least one of the appropriate notification message and the appropriate recommendation message.

11. The ventilatory system of claim 10, wherein the appropriate notification message comprises an alert that the auto-trigger is implicated and information regarding the processed ventilator parameter data that implicated the auto-trigger.

12. The method of claim 10, further comprising:
determining one or more ventilatory settings associated with a ventilatory treatment of the patient wherein the one or more ventilatory settings is a patient interface type;
identifying at least one secondary condition, wherein the at least one secondary condition includes determining if a leak is detected, and
wherein the determining the appropriate recommendation message is based at least in part on the patient interface type and the at least one secondary condition.

13. The ventilatory system of claim 12, wherein the appropriate recommendation message comprises a primary recommendation message and a secondary recommendation message,
wherein the primary recommendation message comprises one of:
a recommendation to adjust trigger sensitivity to a less sensitive setting;
a recommendation to enable leak compensation; and
a recommendation to change a trigger type, and
wherein the secondary recommendation message comprises one of:
a recommendation to check the patient circuit for condensate; and
a recommendation to check the seal of the patient interface.

14. A ventilator-implemented method for detecting asynchrony during ventilation of a patient, the method comprising:
collecting data associated with ventilatory parameters;
processing the collected ventilatory parameter data, wherein the processing the collected ventilatory parameter data comprises deriving ventilatory parameter data from the collected ventilatory parameter data to determine at least one of a pressure integral during exhalation for a moving time window and a flow integral during exhalation for a moving time window;
determining that an ineffective effort is implicated upon detecting that the processed ventilator parameter data breaches a received predetermined threshold; and
issuing a smart prompt when the ineffective effort is implicated.

15. The method of claim 14, wherein the processed ventilator parameter data is the pressure integral during exhalation for a 200 ms moving time window, and
wherein the received predetermined threshold is a determined pressure integral during exhalation for a 200 ms moving window that is less than 0.2 cm of H₂O*second.

16. The method of claim 14, wherein the processed ventilator parameter data is the flow integral during exhalation for a 200 ms moving time window, and
wherein the received predetermined threshold is a determined flow integral during exhalation for a 200 ms moving window that is greater than a derived tidal volume based on a predicted body weight of the patient when no trigger is detected.

17. The method of claim 14, wherein the processed ventilator parameter data is the pressure integral during exhalation for a 200 ms moving time window and the flow integral during exhalation for a 200 ms moving time window, and
wherein the received predetermined threshold is a determined pressure integral during exhalation for a 200 ms moving window that is less than 0.2 cm of H₂O*second and a determined flow integral during exhalation for a 200 ms moving window that is greater than a derived tidal volume based on a predicted body weight of the patient when no trigger is detected.

18. The method of claim 14, wherein the collected ventilatory parameter data comprises:
at least one of airway flow and airway pressure.

19. The method of claim 14, further comprising:
determining an appropriate recommendation message for the issued smart prompt based at least in part on identifying at least one secondary condition,
wherein the at least one secondary condition includes determining if an elevated resistance is detected.

20. The method of claim 19, wherein the determining the appropriate recommendation message for the issued smart prompt is based at least in part on identifying one or more ventilatory settings associated with a ventilatory treatment of the patient,
wherein the one or more ventilatory settings is a breath type, and
wherein the appropriate recommendation message includes one of:
a recommendation to shorten an inspiration time by increasing a peak flow setting or by changing a waveform shape to square;
a recommendation to lower a tidal volume setting;
a recommendation to check for causes of increased resistance;
a recommendation to check for a need to suction or deliver a bronchodilator;
a recommendation to reduce an inspiratory sensitivity setting or to change to a more sensitive trigger type;
a recommendation to increase an expiratory sensitivity setting or to increase a rise time setting;
a recommendation to lower a pressure support setting;
a recommendation to correct a leak, to enable leak compensation, or to increase the expiratory sensitivity setting;
a recommendation to lower an inspiratory pressure setting; and
a recommendation to increase the expiratory sensitivity setting.
